SEQUENCE LISTING (6 sequences)

SEQ ID NO: 1 - DNA, 5239 bp, Homo sapiens
ggatccggattcggattagcagcccgggaagagtgccgtggcacaggcgccggagggagcgcgaccctcggaccccgcctggcccgcggggctgggacccggccccggcctgcccgatggggcgcgcggccccggagatgcgccctcgcccggccccgcgcccccggccccgcgcccccggcccgcccgccccggcccgcgcctccgcctgagtcccccgcgccttggcccgccaccccccgccccgcgcccccggcccgcctgcgccatggagcccggccgcggcggcacagagaccgtgggcaagttcgagttctcccgcaaggacctgatcggccacggcgccttcgcggtggtcttcaagggccgccaccgcgagaagcacgatttggaggtcgccgtcaagtgcattaacaagaagaacctcgccaagtctcagacgctgctggggaaggaaatcaaaatcctgaaggaactgaaacatgaaaacatcgtggccctgtacgacttccaggaaatggctaattctgtctacctggttatggagtactgcaacggtggggacctggccgactacctgcacgccatgcgcacgctgagcgaggacaccatcaggctcttcctgcagcagatcgcgggcgccatgcggcttctgcacagcaaaggcatcatccaccgcgacctgaaaccgcagaacatcctgctgtccaaccccgccggccgccgcgccaaccccaacagcatccgcgtcaagatcgctgacttcggcttcgcgcggtacctccagagcaacatgatggcggccacactctgcggctcccccatgtacatggcccccgaggtcatcatgtcccagcactacgacgggaaggcggacctgtggagcatcggcaccatcgtctaccagtgcctgacggggaaggcgcccttccaggccagcagcccccaggacctgcgcctgttctacgagaagaacaagacgttggtccccaccatcccccgggagacctcggccccgctgcggcagctgctcctggccctactgcaacgcaaccacaaggaccgcatggacttcgatgagttttttcatcaccctttcctcgatgccagcccctcggtcaggaaatccccacccgtgcctgtgccctcgtacccaagctcggggtccggcagcagctccagcagcagctccacctcccacctggcctccccgccgtccctgggcgagatgcagcagctgcagaagaccctggcctccccggctgacaccgctggcttcctgcacagctcccgggactctggtggcagcaaggactcttcctgtgacacagacgacttcgtcatggtccccgcgcagtttccaggtgacctggtggctgaggcgcccagtgccaaacccccgccagacagcctgatgtgcagtgggagctcactggtggcctctgcgggcttggagagccacggccggaccccatctccatccccaccctgcagcagctcccccagtccctcaggccgggctggcccgttctccagcagcaggtgcggcgcctctgtccccatcccagtccccacgcaggtgcagaactaccagcgcattgagcgaaacctgcagtcacccacccagttccaaacacctcggtcctctgccatccgcaggtcaggcagcaccagccccctgggctttgcaagggccagcccctcgccccctgcccacgctgagcatggaggcgtcctggccaggaagatgtctctgggtggaggccggccctacacgccatctcctcaagttggaaccatccctgagcggccaggctggagcgggacgccctccccacagggagctgagatgcggggtggcaggtcccctcgtccaggctcctctgcacccgagcactctccccgcacttccgggctgggctgccgcctgcacagcgcccccaacctgtctgacttgcacgtcgtccgccccaagctgcccaaaccccccacggaccccctgggagctgtgttcagcccaccacaggccagccctccccagccgtcccacggcctgcagtcctgccggaacctgcggggctcacccaagctgcccgacttcctgcagcgaaaccccctgccccccatcctgggctcccccaccaaggctgtgccctcctttgacttcccgaagacccccagctcccagaacctgctggccctcctagcccggcagggcgtggtgatgacgccccctcgaaaccggacgctgcccgacctctcggaggtgggacccttccatggtcagccgttgggccctggcctgcggccaggcgaggaccccaagggcccctttggccggtctttcagcaccagccgcctcactgacctgctccttaaggcggcgtttgggacacaagccccggacccgggcagcacggagagcctgcaggagaagcccatggagatcgcaccctcagctggctttggagggagcctgcacccaggagcccgtgctgggggcaccagcagcccttccccggtggtcttcaccgtgggctctcccccgagcgggagcacgcccccccagggcccccgcaccaggatgttctcagcgggccccactggctctgccagctcttctgcccgccacctggtgcctgggccctgcagcgaggccccagcccctgagctccctgctccaggacacggctgcagctttgccgaccccattgctgcgaacctggagggggctgtgaccttcgaggcccccgacctccctgaggagaccctcatggagcaagagcacacggagatcctgcgtggcctgcgcttcacgctgctgttcgtgcagcacgtcctggagatcgcagccctgaagggcagcgccagtgaggcggcggggggccctgagtaccagctgcaggagagtgtggtggccgaccagatcagcctgctgagccgagaatggggcttcgcggaacagctggtgctgtacctgaaggtggccgagctactgtcctccggcctgcaaagtgccatcgaccagatccgggccggcaagctctgcctgtcgtccactgtgaagcaggtggtgcgcaggctgaatgagctgtacaaggccagcgtggtgtcctgccagggcctgagcctgcggctgcagcgcttcttcctggacaagcagcggctcctggaccgcattcacagcatcactgccgagaggctcatcttcagccacgctgtgcagatggtgcagtcggctgccctggacgagatgttccagcaccgtgagggctgcgtcccacgctaccacaaggccctgctgctcctggaggggctgcagcacatgctctcggaccaggccgacatcgagaacgtcaccaagtgcaagctgtgcattgagcggagactctcggcgctgctgactggcatctgtgcctgacctttctggcctggctgggccccccgtcctgccgagccctgcagagtgggctctgtgtgctggctggactcctcgggacaagcccatggcgctgatcgctggtgctgagccctgccctgggccccacggacagtcagcctgccggcctccctgcagctcacggggcagaaccagcacatctggagccacacagcttggggggtgtctcccatcttttacaggtggggatcacagaatttctgcccctccagctgcctggctcagcaggcgtgggtgccaccaccctctagccccagggcagccccggaggacaggcaagggcctgagaccactgccgactcaaagccaaagcgagctcctgcttagggcaggtcagcaggcactgtgcccaggaagagcctgcggcctcggcgtcccccagtctccaggagcctctccctccgagatacccacccagctttgtcaatcacccaagcactttatgcatatagagacagaacctggacctcaccagggactgctgggcagcgattcctggcagtggcctggtgtttgtacatacacatatgcagacacatgccagggccccccaagcccgagcaccggaccacgttgctgcccaggtctggacctcagcgggagaactggctccggggggagtggggccctgcgctagaggcagaggcagttctttgttcaagcgttcctctggggaccggcagcagaggcaccgtgttctctcagccctggatacgtcttgtaatctttcacactttattcctaaaacgtgtcttatttttatgcagctcattttttctttaaaggagaaaacttgtaggtgtttaagaattggttttgggagggcgaggactgggccaggttagaggcagatggcacaggggcgtgtggcgggcgggtgaggctgctttgcacacctgtgttggtggctgtcccctgccgcccctccctgtggcagcagcaggacaggtgtgtgcccagcaccctccctacctgggcctggaagcagatgaggggaatacttcatgcaaagaaaaaagtaacatgtgcaaaagctccccgtccagctttgacagtcagttttgatgtcagctcctcggcagggtaggcctgatgacagccctgtccctccctgcctctgccttgcccaaggccacggagggcatctgcagagaggcctgccttccggattccaggcgggcatgccctgcaaaccccgcctgggcctcccttggtctgcccagccctcggttagccctgcctgaatcagtagatacttgaacgagtccccagtctgcgggaggcagtggtggggccatggacccatgcggggggttccagggtcacacgccacataacagacaaaaatacacacacgtgtgtttttctttgcaatacttgaaatattgccactgtgcttggacttagaagaagaaaatccccgtgacttcttcctcatcaccttgatggctttattctcaccttgtggggcatgtttgtatttattgcttcatggccgactggaatcctgagtcctgggaagctggcactgcggggatcttgcccggtgtcctggtcctcttgcttccgtcgcggccgcatgtgcgtgtgtccaagcaggtcctgggcgcctcaactgctgcccctggttgaatgttctcttgatagtgctggaccctttgtctattttaaagcgaattttgtgtgatttcctgccctttgcgttatattgtataataccaacgtaaggaaataaacctttggaattgttgggctggtgtcaaaaaaaaaaaaaaaaaa

SEQ ID NO: 2 - protein, 1050 aa, Homo sapiens
MEPGRGGTETVGKFEFSRKDLIGHGAFAVVFKGRHREKHDLEVAVKCINKKNLAKSQTLLGKEIKILKELKHENIVALYDFQEMANSVYLVMEYCNGGDLADYLHAMRTLSEDTIRLFLQQIAGAMRLLHSKGIIHRDLKPQNILLSNPAGRRANPNSIRVKIADFGFARYLQSNMMAATLCGSPMYMAPEVIMSQHYDGKADLWSIGTIVYQCLTGKAPFQASSPQDLRLFYEKNKTLVPTIPRETSAPLRQLLLALLQRNHKDRMDFDEFFHHPFLDASPSVRKSPPVPVPSYPSSGSGSSSSSSSTSHLASPPSLGEMQQLQKTLASPADTAGFLHSSRDSGGSKDSSCDTDDFVMVPAQFPGDLVAEAPSAKPPPDSLMCSGSSLVASAGLESHGRTPSPSPPCSSSPSPSGRAGPFSSSRCGASVPIPVPTQVQNYQRIERNLQSPTQFQTPRSSAIRRSGSTSPLGFARASPSPPAHAEHGGVLARKMSLGGGRPYTPSPQVGTIPERPGWSGTPSPQGAEMRGGRSPRPGSSAPEHSPRTSGLGCRLHSAPNLSDLHVVRPKLPKPPTDPLGAVFSPPQASPPQPSHGLQSCRNLRGSPKLPDFLQRNPLPPILGSPTKAVPSFDFPKTPSSQNLLALLARQGVVMTPPRNRTLPDLSEVGPFHGQPLGPGLRPGEDPKGPFGRSFSTSRLTDLLLKAAFGTQAPDPGSTESLQEKPMEIAPSAGFGGSLHPGARAGGTSSPSPVVFTVGSPPSGSTPPQGPRTRMFSAGPTGSASSSARHLVPGPCSEAPAPELPAPGHGCSFADPITANLEGAVTFEAPDLPEETLMEQEHTEILRGLRFTLLFVQHVLEIAALKGSASEAAGGPEYQLQESVVADQISLLSREWGFAEQLVLYLKVAELLSSGLQSAIDQIRAGKLCLSSTVKQVVRRLNELYKASVVSCQGLSLRLQRFFLDKQRLLDRIHSITAERLIFSHAVQMVQSAALDEMFQHREGCVPRYHKALLLLEGLQHMLSDQADIENVTKCKLCIERRLSALLTGICA

SEQ ID NO: 3 - DNA, 21 bp, Artificial Sequence
  source: /note=“Description of Artificial Sequence Synthetic oligonucleotide”
accgcggcggcacagagaccg

SEQ ID NO: 4 - DNA, 21 bp, Artificial Sequence
  source: /note=“Description of Artificial Sequence Synthetic oligonucleotide”
accgccacggcgccttcgcgg

SEQ ID NO: 5 - DNA, 20 bp, Artificial Sequence
  source: /note=“Description of Artificial Sequence Synthetic oligonucleotide”
agaccaaagcgaaggcgccg

SEQ ID NO: 6 - DNA, 20 bp, Artificial Sequence
  source: /note=“Description of Artificial Sequence Synthetic oligonucleotide”
cggcccgggatcccccgccc

(19) United States
(12) Patent Application Publication
(10) Pub. No.: US 2020/0370056 A1
(43) Pub. Date: Nov. 26, 2020

(54) CELL LINES AND METHODS FOR INCREASED PROTEIN PRODUCTION

(71) Applicant: ASTRAZENECA AB, SODERTALJE (SE)

(72) Inventors: ROBERT ROTH, SODERTALJE (SE); LORENZ MAYR, CAMBRIDGE (GB)

(21) Appl. No.: 16/652,714

(22) PCT Filed: Oct. 2, 2018
(86) PCT No.: PCT/US2018/053860

§ 371 Date: Apr. 1, 2020

Related U.S. Application Data
(60) Provisional application No. 62/566,681, filed on Oct. 2, 2017.

(51) Int. Cl. C12N 15/67

(52) U.S. Cl. CPC C12N 15/67

(57) ABSTRACT

The present technology relates generally to methods for increasing recombinant protein production in various cells lines. In embodiments, impairment of the effects of the ULK1 gene are carried out, resulting in an increase in protein production from a cell line. Also provided are cell lines having increased protein production and methods of preparing such cell lines.

### SEQUENCE LISTING

The instant application contains a Sequence Listing which has been submitted electronically in ASCII format and is hereby incorporated by reference in its entirety. Said ASCII copy, created on Sep. 28, 2018, is named 0098-0001WOI_SL.txt and is 17,225 bytes in sizes.

### FIELD OF THE INVENTION

The present technology relates generally to methods for increasing recombinant protein production in various cells lines. In embodiments, impairment of the effects of the ULK1 gene are carried out, resulting in an increase in protein production from a cell line. Also provided are cell lines having increased protein production, and methods of preparing such cell lines.

### BACKGROUND OF THE INVENTION

As biopharmaceuticals become more and more important in medicine, there is a need to provide increased therapeutic protein yield from various cell types, including eukaryotic cells, such as mammalian cells. This also relates to a desire for high and stably expressing recombinant cell lines, in particular mammalian cell lines.

The generation of recombinant cell clones for production of therapeutic proteins generally requires extensive screening of individual clones to detect and isolate high expressing clones. However, even if high expressing clones are identified in the course of the screening process, these initially high expressing clones often lose their advantageous expression characteristics and the expression yield decreases over time. Therefore, care must be taken in order to identify within a population of successfully expressing cells, those cells that also have a high production stability during prolonged cultivation and therefore, and are not prone to a gradual loss of recombinant protein expression. Therefore, the generation of recombinant cell clones for production of therapeutic proteins and other recombinant polypeptides that are produced on large scale usually comprises excessive and time-consuming screening of individual clones in order to identify the high expressing cell clones that also show the expression stability necessary for large scale production.

What is needed therefore, is a method for producing high volumes of recombinant proteins from various cells lines, as well as methods for generating such cells lines.

### BRIEF SUMMARY OF THE INVENTION

In view of the foregoing, provided herein are methods for impairing the effect of genes, including the ULK1 gene, which translate to increased protein production. Methods are also provided for preparing cells lines for increased protein production, as well as methods for identifying genes that can be impaired and thereby result in increased protein production.

Embodiments hereof are directed to an isolated cell in which an effect of an expression product of a ULK1 gene has been impaired.

Exemplary cells include eukaryotic cells, including mammalian cells, such as Human Embryonic Kidney (HEK) cells and Chinese Hamster Ovary (CHO) cells.

In embodiments, the effect of the expression product of the ULK1 gene is impaired by mutating or editing the ULK1 gene in the isolated cell. Suitably the mutation or edit eliminates or impairs one or more catalytic residues of the expression product of the ULK1 gene, or one or more residues of the expression product of the ULK1 gene that are post translationally modified. In additional embodiments, the mutation or edit reduces the expression of, or knocks out, the ULK1 gene in the cell.

In further embodiments, the effect of the expression product of the ULK1 gene is impaired by post-transcriptional gene interference, which can include siRNA interference, microRNA interference, antisense RNA interference, or small molecule interference.

In embodiments, the impairment of the effect of the expression product of the ULK1 gene results in an increase in production of a recombinant protein as compared to a cell in which the effect of the expression product of the ULK1 gene, has not been impaired. Suitably, the increase in production of the recombinant protein is at least about 30%, and in other embodiments, the increase in production of the recombinant protein is about 50% to about 500%.

Suitably, the recombinant protein is a secreted protein, a membrane-anchored protein, or an intracellular protein. In embodiments, the recombinant protein is an antibody.

Also provided herein are methods of producing a recombinant protein. Such methods include introducing a recombinant gene encoding the recombinant protein into an isolated cell in which an effect of an expression product of a ULK1 gene has been impaired. The isolated cell is cultured under conditions that allow for expression of the recombinant protein. The recombinant protein is then isolated from the isolated cell or from the culture medium if the recombinant protein is secreted by the isolated cell.

Also provided are methods of producing an isolated cell for use in recombinant protein production, which include impairing an effect of an expression product of a ULK1 gene in the isolated cell and culturing the isolated cell under conditions that allow for expansion of the isolated cell.

In still further embodiments, provided herein are methods of screening for a gene, an expression product of which when impaired, results in increased production of a recombinant protein. The methods include impairing a function of the expression product of the an isolated cell to create an impaired isolated cell, and introducing a recombinant gene encoding the recombinant protein into the impaired isolated cell. The impaired isolated cell is then cultured under conditions that allow for expression of the recombinant protein, and the recombinant protein is isolated from the impaired isolated cell. The volume of production of the recombinant protein is determined, and compared to the volume of production of the recombinant protein in a cell in which the effect of the expression product of the gene has not been impaired. An increase in the volume of productionof the recombinant protein in the impaired isolated cell of at least about 30% is indicative that the gene, the expression product of which, when impaired, results in increased production of recombinant protein.

### BRIEF DESCRIPTION OF DRAWINGS

The foregoing and other features and aspects of the present technology can be better understood from the following description of embodiments and as illustrated in the accompanying drawings. The accompanying drawings, which are incorporated herein and form a part of the specification, further serve to illustrate the principles of the present technology. The components in the drawings are not necessarily to scale.

FIG. 1 shows a flowchart of an experimental design for screening for genes of interest, in accordance with embodiments described herein.

FIG. 2 shows a flow cylornetry analysis of Expi293F cells, expressing three different model proteins.

FIG. 3A shows a flow cytometry analysis of Expi293F cells demonstrating different levels of protein expression.

FIG. 3B shows an SDS-page illustrating different levels of Cripto-Fc protein production in Expi293F cells.

FIG. 4 shows screening data across 24 wells in a primary screening in accordance with embodiments hereof.

FIG. 5 shows a pie chart illustrating the outcome of confirmation screening of the primary hits that were active on the CriptoFC cell line on the cell lines expressing PLAP and GFRa2.

FIG. 6 shows cell viability when treated with various compounds using a recombinant protein expression assay as described herein.

FIG. 7 shows enzymatic activity when cells are treated with various compounds, using a recombinant protein assay as described herein.

FIG. 8 shows volume of protein produced in a PLAP cell assay.

FIGS. 9A and 9B show the results of gene editing experiments on the ULK1 gene in HEK293 cells expressing Cripto-Fc.

FIG. 10A shows the results of siRNA down regulation of the ULK1 gene in Expi293-Cripto-Fc cells.

FIG. 10B shows the extent of mRNA knock down achieved siRNA.

FIG. 11. shows a flow cytometry analysis of Expi293F cells demonstrating different levels of protein expression after genetic impairment of ULK1 activity.

FIGS. 12A-12C show the effect of cell culture parameters on a CHO cell line after treatment with an ULK1 inhibitor.

FIGS. 12D-12E show the volumetric and specific productivity or Ab001 in a CHO cell line measured by Protein A analyses after treatment with an ULK1 inhibitor.

### DETAILED DESCRIPTION OF THE INVENTION

It should be appreciated that the particular implementations shown and described herein are examples and are not intended to othenvise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase specifically taught in this specification shall he resolved in favor of the latter.

As used in this specification, the singular forms “a,”“an” and “the” specificially also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of skill in the art.

The methods, processes, cells and composition described herein are based on the surprising finding that cells (including eukaryotic cells, such as mammalian cells), in which the effect of the expression product of the ULK1 gene is impaired, are capable of expressing as recombinant protein product of interest with significantly improved yield.

### Isolated Cells

In embodiments, provided herein is an isolated cell in which an effect of an expression product of a ULK1 gene has been impaired.

The mRNA nucleotide sequence of the human ULK1 gene is provided below as SEQ ID NO:1.

NM_003565.2 Homo sapiens unc-51 like autophagy activating kinase 1
(ULK1), mRNA
(SEQ ID NO: 1)
GGATCCGGATTCGGATTAGCAGCCCGGGAAGAGTGCCGTGGCACAGGCGCCGGAGGGAGCGCGACCCTCG
GACCCCGCCTGGCCCGCGGGGCTGGGACCCGGCCCCGGCCTGCCCGATGGGGCGCGCGGCCCCGGAGATG
CGCCCTCGCCCGGCCCCGCGCCCCCGGCCCCGCGCCCCCGGCCCGCCCGCCCCGGCCCGCGCCTCCGCCT
GAGTCCCCCGCGCCTTGGCCCGCCACCCCCCGCCCCGCGCCCCCGGCCCGCCTGCGCCATGGAGCCCGGC
CGCGGCGGCACAGAGACCGTGGGCAAGTTCGAGTTCTCCCGCAAGGACCTGATCGGCCACGGCGCCTTCG
CGGTGGTCTTCAAGGGCCGCCACCGCGAGAAGCACGATTTGGAGGTCGCCGTCAAGTGCATTAACAAGAA
GAACCTCGCCAAGTCTCAGACGCTGCTGGGGAAGGAAATCAAAATCCTGAAGGAACTGAAACATGAAAAC
ATCGTGGCCCTGTACGACTTCCAGGAAATGGCTAATTCTGTCTACCTGGTTATGGAGTACTGCAACGGTG
GGGACCTGGCCGACTACCTGCACGCCATGCGCACGCTGAGCGAGGACACCATCAGGCTCTTCCTGCAGCA
GATCGCGGGCGCCATGCGGCTTCTGCACAGCAAAGGCATCATCCACCGCGACCTGAAACCGCAGAACATC
CTGCTGTCCAACCCCGCCGGCCGCCGCGCCAACCCCAACAGCATCCGCGTCAAGATCGCTGACTTCGGCT
TCGCGCGGTACCTCCAGAGCAACATGATGGCGGSCACACTCTGCGGCTCCCCCATGTACATGGCCCCCGA
GGTCATCATGTCCCAGCACTACGACGGGAAGGCGGACCTGTGGAGCATCGGCACCATCGTCTACCAGTGC
CTGACGGGGAAGGCGCCCTTCCAGGCCAGCAGCCCCCAGGACCTGCGCCTGTTCTACGAGAAGAACAAGA
CGTTGGTCCCCACCATCCCCCGGGAGACCTCGGCCCCGCTGCGGCAGCTGCTCCTGGCCCTACTGCAACG
CAACCACAAGGACCGCATGGACTTCGATGAGTTTTTTCATCACCCTTTCCTCGATGCCAGCCCCTCGGTC
AGGAAATCCCCACCCGTGCCTGTGCCCTCGTACCCAAGCTCGGGGTCCGGCAGCAGCTCCAGCAGCAGCT
CCACCTCCCACCTGGCCTCCCCGCCGTCCCTGGGCGAGATGCAGCAGCTGCAGAAGACCCTGGCCTCCCC
GGCTGACACCGCTGGCTTCCTGCACAGCTCCCGGGACTCTGGTGGCAGCAAGGACTCTTCCTGTGACACA
GACGACTTCGTCATGGTCCCCGCGCAGTTTCCAGGTGACCTGGTGGCTGAGGCGCCCAGTGCCAAACCCC
CGCCAGACAGCCTGATGTGCAGTGGGAGCTCACTGGTGGCCTCTGCGGGCTTGGAGAGCCACGGCCGGAC
CCCATCTCCATCCCCACCCTGCAGCAGCTCCCCCAGTCCCTCAGGCCGGGCTGGCCCGTTCTCCAGCAGC
AGGTGCGGCGCCTCTGTCCCCATCCCAGTCCCCACGCAGGTGCAGAACTACCAGCGCATTGAGCGAAACC
TGCAGTCACCCACCCAGTTCCAAACACCTCGGTCCTCTGCCATCCGCAGGTCAGGCAGCACCAGCCCCCT
GGGCTTTGCAAGGGCCAGCCCCTCGCCCCCTGCCCGCGCTGAGCATGGAGGCGTCCTGGCCAGGAAGATG
TCTCTGGGTGGAGGCCGGCCCTACACGCCATCTCCTCAAGTTGGAACCATCCCTGAGCGGCCAGGCTGGA
GCGGGACGCCCTCCCCACAGGGAGCTGAGATGCGGGGTGGCAGGTCCCCTCGTCCAGGCTCCTCTGCACC
CGAGCACTCTCCCCGCACTTCCGGGCTGGGCTGCCGCCTGCACAGCGCCCCCAACCTGTCTGACTTGCAC
GTCGTCCGCCCCAAGCTGCCCAAACCCCCCACGGACCCCCTGGGAGCTGTGTTCAGCCCACCACAGGCCA
GCCCTCCCCAGCCGTCCCACGGCCTGCAGTCCTGCCGGAACCTGCGGGGCTCACCCAAGCTGCCCGACTT
CCTGCAGCGAAACCCCCTGCCCCCCATCCTGGGCTCCCCCACCAAGGCTGTGCCCTCCTTTGACTTCCCG
AAGACCCCCAGCTCCCAGAACCTGCTGGCCCTCCTAGCCCGGCAGGGCGTGGTGATGACGCCCCCTCGAA
ACCGGACGCTGCCCGACCTCTCGGAGGTGGGACCCTTCCATGGTCAGCCGTTGGGCCCTGGCCTGCGGCC
AGGCGAGGACCCCAAGGGCCCCTTTGGCCGGTCTTTCAGCACCAGCCGCCTCACTGACCTGCTCCTTAAG
GCGGCGTTTGGGACACAAGCCCCGGACCCGGGCAGCACGGAGAGCCTGCAGGAGAAGCCCATGGAGATCG
CACCCTCAGCTGGCTTTGGAGGGAGCCTGCACCCAGGAGCCCGTGCTGGGGGCACCAGCAGCCCTTCCCC
GGTGGTCTTCACCGTGGGCTCTCCCCCGAGCGGGAGCACGCCCCCCCAGGGCCCCCGCACGAGGATGTTC
TCAGCGGGCCCCACTGGCTCTGCCAGCTCTTCTGCCCGCCACCTGGTGCCTGGGCCCTGCAGCGAGGCCC
CAGCCCCTGAGCTCCCTGCTCCAGGACACGGCTGCAGCTTTGCCGACCCCATTGCTGCGAACCTGGAGGG
GGCTGTGACCTTCGAGGCCCCCGACCTCCCTGAGGAGACCCTCATGGAGCAAGAGCACACGGAGATCCTG
CGTGGCCTGCGCTTCACGCTGCTGTTCGTGCAGCACGTCCTGGAGATCGCAGCCCTGAAGGGCAGCGCCA
GTGAGGCGGCGGGGGGCCCTGAGTACCAGCTGCAGGAGAGTGTGGTGGCCGACCAGATCAGCCTGCTGAG
CCGAGAATGGGGCTTCGCGGAACAGCTGGTGCTGTACCTGAAGGTGGCCGAGCTACTGTCCTCCGGCCTG
CAAAGTGCCATCGACCAGATCCGGGCCGGCAAGCTCTGCCTGTCGTCCACTGTGAAGCAGGTGGTGCGCA
GGCTGAATGAGCTGTACAAGGCCAGCGTGGTGTCCTGCCAGGGCCTGAGCCTGCGGCTGCAGCGCTTCTT
CCTGGACAAGCAGCGGCTCCTGGACCGCATTCACAGCATCACTGCCGAGAGGCTCATCTTCAGCCACGCT
GTGCAGATGGTGCAGTCGGCTGCCCTGGACGAGATGTTCCAGCACCGTGAGGGCTGCGTCCCACGCTACC
ACAAGGCCCTGCTGCTCCTGGAGGGGCTGCAGCACATGCTCTCGGACCAGGCCGACATCGAGAACGTCAC
CAAGTGCAAGCTGTGCATTGAGCGGAGACTCTCGGCGCTGCTGACTGGCATCTGTGCCTGACCTTTCTGG
CCTGGCTGGGGCCCCCCGTCCTGCCGAGCCCTGCAGATGGGCTCTGTGTGCTGGCTGGACTCCTCGGGAC
AAGCCCATGGCGCTGATCGCTGGTGCTGAGCCCTGCCCTGGGCCCCACGGACAGTCAGCCTGCCGGCCTC
CCTGCAGCTCACGGGGCAGAACCAGCACATCTGGAGCCACACAGCTTGGGGGGTGTCTCCCATCTTTTAC
AGGTGGGGATCACAGAATTTCTGCCCCTCCAGCTGCCTGGCTCAGCAGGCGTGGGTGCCACCACCCTCTA
GCCCCAGGGCAGCCCCGGAGGACAGGCAAGGGCCTGAGACCACTGCCGACTCAAAGCCAAAGCGAGCTCC
TGCTTAGGGCAGGTCAGCAGGCACTGTGCCCAGGAAGAGCCTGCGGCCTCGGCGTCCCCCAGTCTCCAGG
AGCCTCTCCCTCCGAGATACCCACCCAGCTTTGTCAATCACCCAAGCACTTTATGCATATAGAGACAGAA
CCTGGACCTCACCAGGGACTGCTGGGCAGCGATTCCTGGCAGTGGCCTGGTGTTTGTACATACACATATG
CAGACACATGCCAGGGCCCCCCAAGCCCGAGCACCGGACCACGTTGCTGCCCAGGTCTGGACCTCAGCGG
GAGAACTGGCTCCGGGGGGAGTGGGGCCCTGCGCTAGAGGCAGAGGCAGTTCTTTGTTCAAGCGTTCCTC
TGGGGACCGGCAGCAGAGGCACCGTGTTCTCTCAGCCCTGGATACGTCTTGTAATCTTTCACACTTTATT
CCTAAAACGTGTCTTATTTTTATGCAGCTCATTTTTTCTTTAAAGGAGAAAACTTGTAGGTGTTTAAGAA
TTGGTTTTGGGAGGGCGAGGACTGGGCCAGGTTAGAGGCAGATGGCACAGGGGCGTGTGGCGGGCGGGTG
AGGCTGCTTTGCACACCTGTGTTGGTGGCTGTCCCCTGCCGCCCCTCCCTGTGGCAGCAGCAGGACAGGT
GTGTGCCCAGCACCCTCCCTACCTGGGCCTGGAAGCAGATGAGGGGAATACTTCATGCAAAGAAAAAAGT
AACATGTGCAAAAGCTCCCCGTCCAGCTTTGACAGTCAGTTTTGATGTCAGCTCCTCGGCAGGGTAGGCC
TGATGACAGCCCTGTCCCTCCCTGCCTCTGCCTTGCCCAAGGCCACGGAGGGCATCTGCAGAGAGGCCTG
CCTTCCGGATTCCAGGCGGGCATGCCCTGCAAACCCCGCCTGGGCCTCCCTTGGTCTGCCCAGCCCTCGG
TTAGCCCTGCCTGAATCAGTAGATACTTGAACGAGTCCCCAGTCTGCGGGAGGCAGTGGTGGGGCCATGG
ACCCATGCGGGGGTTCCAGGGTCACACGCCACATAACAGACAAAATACACACACGTGTGTTTTTCTTT
GCAATACTTGAAATATTGCCACTGTGCTTGGACTTAGAAGAAGAAAATCCCCGTGACTTCTTCCTCATCA
CCTTGATGGCTTTATTCTCACCTTGTGGGGCATGTTTGTATTTATTGCTTCATGGCCGACTGGAATCCTG
AGTCCTGGGAAGCTGGCACTGCGGGGATCTTGCCCGGTGTCCTGGTCCTCTTGCTTCCGTCGCGGCCGCA
TGTGCGTGTGTCCAAGCAGGTCCTGGGCGCCTCAACTGCTGCCCCTGGTTGAATGTTCTCTTGATAGTGC
TGGACCCTTTGTCTATTTTAAAGCGAATTTTGTGTGATTTCCTGCCCTTTGCGTTATATTGTATAATACC
AACGTAAGGAAATAAACCTTTGGAATTGTTGGGCTGGTGTCAAAAAAAAAAAAAAAAAA

The amino sequence of human ULK1 protein is provided below as SEQ ID NO:2.

sp|O75385|ULK1_HUMAN
Serine/threonine-protein kinase ULK1 OS =
Homo sapiens GN = ULK1 PE = 1 SV = 2
(SEQ ID NO: 2)
MEPGRGGTETVGKFEFSRKDLIGHGAFAVVFKGRHRE
KHDLEVAVKCINKKNLAKSQTLLGKEIKILKELKHEN
IVALYDFQEMANSVYLVMEYCNGGDLADYLHAMRTLSE
DTIRLFLQQIAGAMRLLHSKGIIHRDLKPQNILLSNP
AGRRANPNSIRVKIADFGFARYLQSNMMAATLCGSPM
YMAPEVIMSQHYDGKADLWSIGTIVYQCLTGKAPFQA
SSPQDLRLFYEKNKTLVPTIPRETSAPLRQLLLALLQ
RNHKDRMDFDEFFHHPFLDASPSVRKSPPVPVPSYPS
SGSGSSSSSSSTSHLASPPSLGEMQQLQKTLASPADT
AGFLHSSRDSGGSKDSSCDTDDFVMVPAQFPGDLVAE
APSAKPPPDSLMCSGSSLVASAGLESHGRTPSPSPPC
SSSPSPSGRAGPFSSSRCGASVPIPVPTQVQNYQRIE
RNLQSPTQFQTPRSSAIRRSGSTSPLGFARASPSPPA
HAEHGGVLARKMSLGGGRPYTPSPQVGTIPERPGWSG
TPSPQGAEMRGGRSPRPGSSAPEHSPRTSGLGCRLHS
APNLSDLHVVRPKLPKPPTDPLGAVFSPPQASPPQPS
HGLQSCRNLRGSPKLPDFLQRNPLPPILGSPTKAVPS
FDFPKTPSSQNLLALLARQGVVMTPPRNRTLPDLSEV
GPFHGQPLGPGLRPGEDPKGPFGRSFSTSRLTDLLLK
AAFGTQAPDPGSTESLQEKPMEIAPSAGFGGSLHPGA
RAGGTSSPSPVVFTVGSPPSGSTPPQGPRTRMFSAGP
TGSASSSARHLVPGPCSEAPAPELPAPGHGCSFADPI
TANLEGAVTFEAPDLPEETLMEQEHTEILRGLRFTLL
FVQHVLEIAALKGSASEAAGGPEYQLQESVVADQISL
LSREWGFAEQLVLYLKVAELLSSGLQSAIDQIRAGKL
CLSSTVKQVVRRLNELYKASVVSCQGLSLRLQRFFLD
KQRLLDRIHSITAERLIFSHAVQMVQSAALDEMFQHR
EGCVPRYHKALLLLEGLQHMLSDQADIENVTKCKLCI
ERRLSALLTGICA

In addition to targeting impairment of the ULK1 gene, the methods described herein can be utilized to target and impair the expression product of various genes that are involved in recombinant protein production. As described herein, it is desirable to target genes, the impairment of which (or the impairment of their expression products), not only provide increased protein expression, but also do not negtitively impact cell survival, allowing for the greatest possibility of protein recovery, isolation, and eventually use and composition formulation.

In establishing genes that can be targeted to increase protein production in a cell, as described herein, it is desirable to target processes triggered during unfolding protein response (UPR) of a cell, but which have a limiting effect on the output through the secretory machinery, while maintaining stress responses. The UPR of a cell during stress expands the endoplasmic reticulum (ER) several fold to temporarily increase the capacity of the ER. The expansion of the ER is counteracted by autophagy that degrades organelles in a controlled manner, in order to maintain cell homeostasis.

The ULK1 gene encodes the serine/threonin-protein kinase ULK1. It is involved in autophagy in a cell in response to starvation. The ULK1 protein acts upstream of phosphatidylinositol 3-kinase PIK3C3 to regulate the formation of autophagophores, the precursors of autophagosomes. It is part of regulatory feedback loops in autophagy, acting as both as a downstream effector and negative regulator of mammalian target of rapamycin complex 1 (mTORC1) via interaction with RPTOR. It has also been identified to be involved with sorting vesicles exiting the endoplasmic reticulum (ER), and in determining if the vesicle destination is Golvi degradative lysozomes.

Based on these activities, it was determined as described herein, that impairing the effect of the expression product of the ULK1 gene increases the production of recombinant proteins by a cell, most likely by inhibition degradation of ER during UPR, and by preventing funneling of secretory vesicles to degradation pathways. However, impairing the effect of the ULK1 gene expression product does not negatively impact cellular growth to any great extent.

As used herein “ULK1 gene” refers to any endogenous gene which encodes a protein that shares at least 50%, at least 60%, at least 70%, at least 75%, at least 80%, at least 85%, at least 90%, at least 95%, at least 96%, at least 97%, at least 98% or at least 99% homology or identity to the amino acid sequence shown in SEQ ID NO: 2, or the protein encoded by the nucleic acid of SEQ ID NO 1. The protein encoded by such gene preferably has the same function as the protein having an amino acid sequence as is shown in SEQ ID NO: 2, or the protein encoded by the nucleic acid of SEQ ID NO: 2. Said gene can be modified as described herein in order to impair the function of the expression product that is expressed by the unmodified cell. The term “ULK1 gene” encompasses coding and non-coding regions of the as well as promoter region(s) and 5′-untranslated regoins of the gene.

The terms “ULK1 protein” or “expression product of the ULK1 gene” and similar expressions as used herein, encompass homologs and orthologs of ULK1 and in particular encompass any protein that shares at least 50%, at least 60%, at least 70%, at least 75%, at least 80%, at least 85%, at least 90%, at least 95%, at least 96%, at least 97%, at least 98% or at least 99% homology or identity to the amino acid sequence shown in SEQ ID NO: 2 or the protein encoded by SEQ ID NO 1. Such proteins suitably have the same function as the protein having an amino acid sequence as is shown in SEQ ID NO: 2, or the protein encoded by the nucleic acid of SEQ ID NO: 1. Homology, respectively identity, may be calculated over the entire length of the protein.

As used herein, the amount of “production or a protein,” or “protein production,” or “production of a recombinant protein” means the volumetric production or volumetric productivity, of a cell or a cell line, generally measured in mg of protein/mL of cell culture (mg/mL).

As described herein, the effect of the expression product of the ULK1 gene can be impaired by various mechanisms or actions, thereby lowering, reducing or eliminating the functional expression of the endogenous gene UKL1, by deleting or knocking-out the ULK1 gene, or by introducing mutations into the ULK1 gene. In addition the expression product can be impaired by eliminating or impairing the action of the expression product of the ULK1 gene, or otherwise counteracting the activity of the expression product of the ULK1 gene.

As described herein, the effect of the expression product of the ULK1 gene, and hence the ULK1 protein, may be impaired e.g. on the gene level or on the protein level. The effect of ULK1 can be impaired, for example, by modification of the structure/sequence of the ULK1 protein, the transcription and/or translation.

For example, the effect of the expression product of the ULK1 gene can be impaired in a cell because functional expression of the ULK1 gene is reduced or eliminated in the cell. Altering the expression of the ULK1 gene, e.g. by gene silencing or by deleting said gene is an exemplary method to provide cells capable of expressing a recombinant product of interest with high yield. When the expression level of the ULK1 gene is reduced or eliminated in a cell and hence, less or no functional ULK1 protein is produced by the respectively altered cell, the expression yield of a recombinant product (protein) of interest is increased. This correlation between functional ULK1 expression and yield of recombinant protein expression is an unexpected finding.

Lowering, reducing, or eliminating (no functional expression) functional expression of the ULK1 gene may be achieved, for example, by reducing the expression level of the ULK1 gene or by disrupting the function of the expression product of ULK1 or by a combination of such methodologies.

In an exemplary embodiment, a cell can be altered so that functional expression of the ULK1 gene is lowered, reduced or eliminated by gene knock-out, gene mutation, gene deletion, gene editing, gene silencing or a combination of any of the foregoing. That is, the mutation or gene edit reduces the expression, lowers the expression, or eliminates (knocks out) the expression of the ULK1 gene and/or the ULK1 gene product (protein).

According to one embodiment, functional expression of the ULK1 gene can be reduced or eliminated in a cell by gene knockout. A gene knockout is a genetic technique by which a gene is made inoperative by disrupting its function. For example, a nucleic acid can be inserted into the coding sequence, thereby disrupting the gene function. Furthermore, the complete ULK1 gene or a portion thereof can be deleted, whereby no or no functional protein is expressed by the respectively altered cell. Additional embodiments can introduce one or more knock-out mutations into the coding sequence, which renders a non- or a less functional expression product, one or more frameshift mutations can be introduced that result in a non- or less-functional protein. Alternatively or additionally, one or more stop codons can be introduced into the coding sequence so that a truncated, non- or less functional protein is obtained. Hence, according to one embodiment, the ULK1 gene comprises one or more mutations which provide a non- or less functional expression product. Other options include but are not limited to one or more mutations in the promoter, in the 5′- and/or 3′ untranslated region (UTR) or other regulatory elements of the ULK gene. According to an embodiment, the promoter function of the ULK1 gene is disrupted, e.g. by introducing a promoter deletion or by introducing a construct between the promoter and the transcription start. Methods for achieving a gene knockout to suppress or eliminate expression of the target gene are well-known to the skilled person, as well as described herein. In additional embodiments, functional expression of the ULK1 gene can be lowered, reduced or eliminated by targeting a regulatory element involved in the regulation of expression of the ULK1 gene, for example transcription factor, promoter, enhancer, UTRs, or other regulatory elements can be targeted e.g. by knock-out, deletion, down-regulation or any other alteration that inactivates or reduces the activity of the regulatory element, thereby preventing or reducing functional expression of the ULK1 gene and thereby impairing the effect of the endogenous expression product of the gene.

In embodiments, mutations in the UKL1 can impair the function or effect of the ULK1 gene by impacting function or activation of the expression product by mutating the active site or ATP binding pocket of the UKL1 gene product. Additional mutations can modify the amino acid sequence of the protein at sites such as those listed below:

TABLE 1
Potential Locations for Impairment of ULK1 Gene Product

| Amino acid | Function | Effect | Modified by |
|---|---|---|---|
| Lys46 | Active site | loss of function | n/a |
| Met92 | Active site | loss of function | n/a |
| Lys162 | Acetylation | activation | TIP60 |
| Thr180 | Phosphorylation | activation | ULK1 |
| Ser317 | Phosphorylation | activation | AMPK |
| Ser467 | Phosphorylation | activation | AMPK |
| Ser555 | Phosphorylation | activation | AMPK |
| Ser575 | Phosphorylation | activation | AMPK |
| Ser637 | Phosphorylation | activation | AMPK |
| Ser757 | Phosphorylation | inhibition | MTOR |
| Ser777 | Phosphorylation | activation | AMPK |

A person of ordinary skill in the art can determine potential nucleic acid mutations in the gene sequence to impair these amino acid locations.

In additional embodiments, a dominant negative mutation (also called antimorphic mutation) can be utilized to impair the ULK-1 gene. Dominant negative mutations result in an altered gene product that acts antagonistically to the wild-type allele. These mutations usually result in an altered molecular function (often inactive) and are characterized by a dominant or semi-dominant phenotype.

In an exemplary embodiment, the ULK1 gene is functionally knocked out by genetic engineering. Examples include but are not limited to genome editing, such as genome editing, with engineered nucleases (GEEN). This is a type of genetic engineering in which DNA is inserted, replaced or removed from a genome using artificially engineered nucleases, or “moleular scissors.” The nucleases create specific double-stranded breaks (DSBs) at desired locations in the genome, and harness the cells endogenous mechanisms to repair the induced break by natural processes of homologous recombination (HR) and nonhomologous end-joining (NHEJ). Exemplary engineered nucleases include Zinc finger nucleases (ZFNs), Transcription Activator-Like Effector Nucleases (TALENs), CRISPR-Cas9, and engineered meganuclease re-engineered homing endonucleases.

Exemplary target sequences for CRISPR-Cas9 gene editing include:

ULK1 gRNA C8
(SEQ ID NO: 3)
ACCGCGGCGGCACAGAGACCG for human ULK1
(281-299 in exon 1)
ULK1 gRNA D8
(SEQ ID NO: 4)
ACCGCCACGGCGCCTTCGCGG for human ULK1
(336-353 in exon 1)

In embodiments, one or more copies of the ULK1 gene present in the genome of a cell are altered, e.g. knocked-out or deleted, to reduce or eliminate and hence impair the effect of the expression product of the ULK1 gene in the cell. Thus, according to one embodiment, at least one copy of the ULK1 gene is deleted or functionally inactivated in the genome of the cell. For example, one or more mutations may be inserted into the copy or copies of the ULK1 gene (if more than one copy is present, to provide a non- or less functional expression product or to eliminate or reduce expression in toto and, hence impair the effect of ULK1 in the cell. According to one embodiment, in case more than one copy is present, all copies of the ULK1 gene are suitably altered in the cell.

In exemplary embodiments, the mutation or gene edit eliminates or impairs one or more catalytic residues of the expression product of the ULK1 gene. A “catalytic residue” refers to one or more sites of a protein structure in which a reaction or binding with the protein occurs or is catalyzed. By mutating or editing one or more of these catalytic residues, a ULK1 gene product that may be produced in a cell will not be able to function normally, resulting in lowered, reduced or fully eliminated function of the ULK1 protein.

In additional embodiments, the mutations or gene editing can impair or one or more residues of the expression product of the ULK1 gene that are post translationally modified. That is, one or more residues of the ULK1 protein can be mutated or edited, resulting in an impaired ULK1 protein product that has lowered, reduced, or completely eliminated, function.

The term “isolated cell” as used herein refers to a cell that is outside of a living organism (e.g., plant, insect or animal). Exemplary cells that can be prepared and utilized in the methods described herein include prokaryotic and eukaryotic cells. As described herein, the cells can be provided as a cell culture, a cell line, cell clone and the like. Exemplary prokaryotic cells include bacteria such as E. coli, Corynebacterium and Pseudomonas fluorescens. Eukaryotic cells include yeast, such as Saccharomyces cerevisiae, insect cells, including cell lines derived from Spodoptera frugiperda, etc. Exemplary eukaryotic cells include mammalian cells such as rodent cells, human cells and monkey cells. Suitable eukaryotic cells are rodent cells such as e.g. cells derived from hamster or mouse. They can be a Chinese hamster cell such as a Chinese Hamster Ovary (CHO) cell, a BHK cell, a NSO cell, a C127 cell, a mouse 3T3 fibroblast cell, and a SP2/0 cell. Examples of CHO cells are CHO-K1, CHO-S, CHO-K1 SV, CHO-SSF3, CHO-DG44, CHO-DUXB1. Additional eukaryonc cell lines include Human Embryonic Kidney (HEK) cells mouse myeloma lymphoblastoid cells, human embryonic retinal cells, and human amniocyte cells, etc.

In order to provide production cell lines with uniform and thus predictable characteristics, it is desirable to alter the genome of the cell (e.g. a eukaryotic cell) to achieve impairment. The respectively altered cells can then be transfected with an expression vector or other carrier comprising a polynucleotide encoding a protein product of interest. For example, reducing or eliminating the expression of the ULK1 gene in a CHO cell provides CHO cells which are capable of producing a recombinant protein product with significantly increased yield. In additional embodiments, a eukaryotic cell derived from a human cell, for example a HEK293 cell, a MCF-7 cell, a PerC6 cell, a CAP cell, hematopoietic cells and a HeLa cell, can be utilized. Another alternative are monkey cells, including COS cells, COS-1, a COS-7 cell and a Vero cell.

As described herein, a cell is modified to impair the effect of the expression product of the ULK1 gene in the cell compared to a corresponding, unmodified cell, which endogenously expresses ULK1. Impairment is achieved by reducing or eliminating functional expression of the ULK1 gene or by impairing the activity or effect of the ULK1 gene product.

According to one embodiment, the deletion of the ULK1 gene can be carried out via a chromosome breakage. A chromosome breakage can be induced e.g. by treating the cells with a toxic agent that promotes chromosome breakage, such as MTX, aphidicolin or hygromycin. Other options for inducing chromosome breakages include but are not limited to radiation, irradiation, mutagens, carcinogenic substances and bleomycin. Chromosome breakages may also occur spontaneously during transfection, for example, electroporation. After inducing chromosome breakage, cells having the desired breakpoint (which results in a deletion of the ULK1 gene) can be identified by analyzing their DNA.

Functional expression of the ULK1 gene can be influenced by various activities, for example by altering the promoter and/or an enhancer of the ULK1 gene so that less or no transcript is produced, or by gene silencing technologies such as transcriptional or post-transcriptional gene silencing. According to one embodiment, an isolated cell can contain one or more mutations in the promoter region of the ULK1 gene. For example, the promoter region may be altered to provide a less functional or non-functional promoter, the promoter may also be completely eliminated. Alternatively or in addition, it is possible to add a polynucleotide sequence encoding a polypeptide including a stop codon between the promoter and the start codon of the ULK1 gene which halts production of the ULK1 polypeptide.

Reduction of functional gene expression may achieve a level where expression is lowered, reduced or even eliminated. Post-transcriptional gene silencing can be achieved by antisense molecules or molecules that mediate RNA interference. As described herein, post-transcriptional gene silencing can utilize siRNA interference, microRNA interference, antisense RNA interference or small molecule interference.

For example, antisense polymadeotides can be designed to specifically bind to the ULK1 gene's transcribed RNA, resulting in the formation of RNA-DNA or RNA-RNA hybrids, with an arrest of reverse transcription or messenger RNA translation. Many forms of antisense have been developed and can be broadly categorized into enzyme-dependent antisense or steric blocking antisense. Enzyme-dependent antisense includes forms dependent on RNase H activity to degrade target mRNA, including single-stranded DNA, RNA, and phosphorothioate antisense. Antisense polynucleotides are typically generated within the cell by expression from antisense constructs that contain the antisense strand as the transcribed strand. Trans-cleaving catalytic RNAs (ribozymes) are RNA molecules possessing endoribonuclease activity. Ribozymes may be specifically designed for a particular target and may be engineered to cleave any RNA species site-specifically in the background of cellular RNA. The cleavage event renders the mRNA unstable and prevents protein expression. The genome of the cell can be altered so that a respective antisense molecule is permanently expressed.

An additional embodiment useful in reducing functional expression of the ULK1 gene on a post transcriptional level is based on RNA interference (RNAi), thereby resulting in increased recombinant protein production from the cell. Methods for silencing genes by RNAi are well known in the art, and include, but are not limited to, short interfering nucleic acids (siNA) short interfering RNA (siRNA), microRNA (miRNA), short hairpin RNAs (shRNA) as well as precursors thereof which are processed in the cell to the actual RNAi inducing compound.

For example, a siRNA can be used for silencing the ULK1 gene. The siRNA can be provided as a double-stranded molecule having 3′ overhangs on each strand. Blunt ended molecules can also be used. The siRNA can comprise desoxy- as well as ribonucleotides and furthermore, can comprise modified nucleotides. Several embodiments and variations of siRNA compounds are known in an and can be used to reduce expression of the ULK1 gene. Exemplary siRNAs targeting regions of the target ULK1 gene on the RNA level can be identified by using proper computational methods, applying certain design-algorithms.

Exemplary siRNA compounds include those listed below:

TABLE 2
Exemplary siRNA Compounds

| | | | | | mRNA |
|---|---|---|---|---|---|
| Brand | Target | Company | Art. No | Exon | position |
| Silencer | ULK1 | Life | s15963 | Exon 8 | 893 |
| Select siRNA | | Technologies | | | |
| Silencer | ULK1 | Life | s15964 | Exon | 1073 |
| Select siRNA | | Technologies | | 10, 11 | |
| Silencer | ULK1 | Life | s15965 | Exon 9 | 964 |
| Select siRNA | | Technologies | | | |

In order to obtain a siRNA against the target transcript, the double-stranded molecule can be transfected directly into the cell. Alternatively, the siRNA may result from processing by dicer, an enzyme that converts either long dsRNAs or small hairpin RNAs (shRNAs) into siRNAs. These precursors or the final siRNA molecules can be produced exogenously (artificially) and can then he introduced into the cells by various transfection methods. According to a further embodiment, the RNAi inducing compound can be expressed by a vector that is transfected into the cell. For sRNA, this can be done by the introduction of a loop between the two strands, thus producing a single transcript, which can be then processed into a functional siRNA in the cell. Such transcription cassettes typically use an RNA polymerase 3 promoter (for example U6 or H1) which usually directs the transcription of small nuclear RNAs (shRNAs). The resulting shRNA transcript from the vector is then processed by dicer, thereby producing the double-stranded siRNA molecules, suitably having the characteristic 3′ overhangs. According to one embodiment, such shRNA providing vector is stably integrated into the genome of the cell. Cells comprising a respective shRNA providing vector can then be transfected with an expression vector comprising a polynticleotide encoding the product of interest. Alternatively, co-transfection strategies can be used, wherein the vector generating the shRNA is co-transfected with the expression vector comprising the polynucleotide encoding the product of interest.

Transcriptional gene silencing can include epigenetic modifications. According to one embodiment, expression of the ULK1 gene is reduced by epigenetic silencing, including DNA methylation. In addition, the sequence of the ULK1 gene can be changed to reduce the half-life of the mRNA. This also achieves a reduction in the effect of the ULK1 protein in the respectively altered cell.

According to an embodiment, the genome of a cell (e.g., a eukatyotic cell) is altered to impair the effect of ULK1 by heterologous expression of a mutant ULK1 which is non- or less functional than the endogenously expressed ULK1 protein. In this embodiment, the isolated cell comprises, in addition to the heterologous polynucleotide encoding the polypeptide of interest, a further heterologous polynucleotide encoding the mutant ULK1. By overexpressing a respective non- or less functional mutation form of ULK1, a dominant negative phenotype can be created. A further option to impair and hence reduce the effect of ULK1 in the cell is the heterologous expression of a protein such as an antibody which neutralizes ULK1 and hence impairs the effect of ULK1 in the cell. According to one embodiment, the effect of ULK1 is unpaired in the cell by reducing or eliminating functional expression of molecules that functionally interact with ULK1.

In additional embodiments, a low molecular weight compound, i.e., small molecule, is used to inhibit expression of the ULK1 gene by specifically inhibiting binding of a transcription factor to a regulatory region in the promoter, or by inhibiting an activator of transcription required for transcription of the target gene or by inhibiting the effect of the expression product of the ULK1 gene directly. Respective inhibitory compounds include, but are not limited to, chemical compounds such as in particular small molecules, proteins and peptides. Another possibility is to use of compounds such as low molecular weight compounds stimulating degradation of the protein product, for example by stimulating ubiquitination of the protein.

Small molecules can also be used to impair translated proteins, for example hy protein destruction or limiting activity. In embodiments, small molecules can function by mediating degradation of a protein, for example using proteolysis-targeting chimera (PROTAC) technology. Intrabody molecules (i.e., intracellular antibodies), can also be used to bind expressed proteins and render them non-functional or to direct their destruction. Exemplary small molecules are described herein and in the Examples, and include compounds such as the ULK1 inhibitor, MRT68921 (IC₅₀ for ULK1 approx: 2.9 nM):

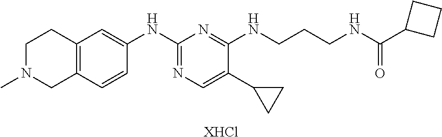

In exemplary embodiments, expression of the ULK1 gene is reduced by at least 3 fold, at least 5 fold, at least 10 fold, at least 20 fold, at least 30 fold, at least 40 fold, at least 50 fold, at least 60 fold, at least 70 fold, at least 75 fold, at least 80 fold, at least 90 fold, at least 100 fold, at least 125 fold, at least 250 fold, at least 500 fold, at least 750 fold, at least 1000 fold, at least 1250 fold, at least 1500 fold, at least 1750 fold or at least 2000 fold, etc. The amount of reduction of expression of the ULK1 gene can be determined, for example, by using real-time RT-PCR or other sensitive RNA detection methods. Such reduction can be measured in comparison to an unmodified reference cell wherein the expression of the ULK1 gene is not reduced.

In additional embodiments, expression of the ULK1 gene can be measured relative to another native gene in the cell, to determine the amount of reduction. For example, expression of the ULK1 gene can be 0.05% or less, 0.0475% or less, 0.045% or less, 0.0425% or less, 0.04% or less, 0.0375% or less, 0.035% or less, 0.0325% or less, 0.03% or less, 0.0275% or less, 0.025% or less, 0.0225% or less, 0.02% or less, 0.0175% or less, 0.015% or less compared to the expression of an 18S ribosomal RNA (set as 100%) in the same cell. According to one embodiment, expression of the ULK1 gene can be even less, such as 0.001% or less, 0.0001 or less or even 0.00001% or less, compared to the expression of the 18S RNA (set as 100%) in the same cell.

The functional expression of the ULK1 gene is suitably impaired, i.e., reduced lowered or eliminated, such that it results in an increase in the expression and production of a recombinant protein product of interest if the modified cell is transfected art expression vector encoding the ptoduct of interest, compared to a corresponding cell wherein the functional expression of the ULK1 gene is not impaired (i.e., not lowered, reduced or eliminated). According to an embodiment, expression of the recombinant protein product of interest is increased by at least about 20%, more suitably at least about 30%, relative to the expression in a corresponding cell wherein the functional expression of the ULK1 gene is not lowered, reduced or eliminated.

Suitably, the expression of a recombinant protein is measured as a volume of protein produced (e.g., mL) using methods known in the art, and thus the increase in production refers to an increase in the volume of protein produced. According to further embodiments, expression of the recombinant protein product of interest is increased by at least about 30%, at least about 40%, at least about 50%, at least about 60%, at least about 70%, at least about 80%, at least about 90%, at least about 100%, at least about 150% at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 100%, or by about 30% to about 700%, about 40% to about 600%, about 50% to about 500%, or about 100% to about 500%, relative to the expression in a corresponding cell wherein the functional expression of the ULK1 gene is not lowered, reduced or eliminated.

A “heterologous polynucleotide” or “heterologous nucleic acid” and likewise expressions used herein, refer to a polynucleotide sequence that has been introduced into the eukaryotic cell, e.g. by the use of recombinant techniques such as transfection. A “polynucleotide” in particular refers to a polymer of nucleotides which are usually linked from one deoxyribose or ribose to another and refers to DNA as well as RNA, depending on the context. The term “polynucleotide” does not comprise any size restrictions.

In certain embodiments, a cell does not comprise a heterologous polynucleotide encoding a product of interest, a heterologous polynucleotide encoding a selectable marker and/or a heterologous polynucleotide encoding a reporter polypeptide that is/are expressed, or secreted from said cell. A respective “empty” cell can be used as a cloning cell line for recombinant production technologies. respective cell can then be transfected with a heterologous polynucleotide encoding a protein product of interest, e.g. using an appropriate expression vector. Such “empty” cells in which the effect of the expression product of the ULK1 gene is impaired and which do not yet express and do not yet secrete a recombinant product, can thus be transfected with different expression vectors, depending on the desired product of interest that is supposed to be recombinantly produced. Thus, such cell lines can used for different projects, i.e. for the production of different products of interest, in particular secreted polypeptides of interest. Transfection can be transient or stable.

As described herein, in suitable embodiments, the cell (e.g., a eukaryotic cell) comprises a heterologous polynucleotide encoding a product of interest. The product of interest is suitably a recombinant product that is to be expressed by the cell in large quantity. Suitably the product of interest is a polypeptide. Furthermore, the cell may additionally comprises a heterologous polynucleotide encoding a selectable marker and/or a heterologous polynucleotide encoding a reporter. This simplifies the selection of host cells which are successfully transfected and thus express the product of interest. Furthermore, the cell may comprise several polynucleotides encoding different selectable markers and/or reporter polypeptides. According to one embodiment, the heterologous polynucleotide encoding the product of interest is stably integrated into the genome of the cell.

An expression vector can be used to introduce heterologous polynucleotides into a cell. The polynucleotides can be comprised in an expression cassette. The polynucleotide(s) encoding the product of interest and the polynucleotide(s) encoding a selectable marker or reporter polypeptide may be located on the same or different expression vectors. Introduction into a cell may be achieved by transfecting a suitable expression vector comprising the polynucleotide encoding the product of interest into the host cells. The expression vector suitably integrates into the genome of the host cell (stable transfection). In case the heterologous nucleic acid is not inserted into the genome, the heterologous nucleic acid can be lost at the later stage, when the cells undergo mitosis (transient transfection). Stable transfection is suitable for generating high expressing cell clones for producing a product of interest such as a polypeptide of interest on industrial scale. Exemplary methods for introducing a heterologous nucleic acid such as an expression vector into a host cell include, but are not limited to, calcium phosphate transfection, electroporation, lipofection, biolistic- and polymer-mediated gene transfer and the like. Recombination mediated approaches can be used to transfer the heterologous polynucleotide into the host cell genome.

Expression vectors used to achieve expression of a recombinant product of interest usually contain transcriptional control elements suitable to drive transcription such as promoters, enhancers, polyadenylation signls, transcription pausing or termination signals usually as elements of an expression cassette. If the desired product is a polypeptide, suitable translational control elements are included in the vector, such as 5′ translated regions leading to 5′ cap structures suitable for recruiting ribosomes and stop codons to terminate the translation process.

In embodiments, polynucleotide(s) encoding the product of interest and polynucleotides encoding a selectable marker(s) and or reporter polypeptide(s) are suitably comprised in expression cassettes. For example, each of said polynucleotide(s) can be comprised in a separate expression cassette. It is also within the scope of the present invention that at least two of the respective polynucleotides are comprised in one expression cassette. According to one embodiment, at least one internal ribosomal entry site (IRES) element is functionally located between the polynucleotides that are expressed from the same expression cassette. Thereby, it is ensured that separate translation products are obtained from said transcript. Respective IRES based expression technologies and other bi- and polycistronic systems are well known in the art.

In embodiments, the expression vector may comprise at least one promoter and/or promoter/enhancer element as an element of an expression cassette. Promoters can be divided in two classes, those that function constitutively and those that are regulated by induction or depression. Both are suitable. Strong constitutive promoters which drive expression in many cell types include, but not limited to, the adenovirus major late promoter, the human cytomegalovirus immediate early promoter, the SV40 and Rous Sarcoma virus promoter, and the murine 3-phosphoglycerate kinase promoter, EF1α. According to one embodiment, the promoter and/or enhancer is either obtained from CMV and/or SV40. The transcription promoters can be selected from the group consistingof an SV40 promoter, a CMV promoter, an EF1 alpha promoter, a RSV promoter, a BROAD3 promoter, a murine rose 26 promoter, a pCEFL promoter and a β-actin promoter.

The expression product of interest can be any biological product capable of being produced hy transcription, translation or any other event of expression of the genetic inthrmation encoded by the polynucleotide encoding the product of interest. The product of interest may be selected from the group consisting of polypeptides and nucleic acids, in particular RNA. The product can be a pharmaceutically or therapeutically active compound, or a research tool to be utilized in assays and the like. Suitably, the product of interest is a polypeptide and in particular a recombinant polypeptide, i.e., a polypeptide that is produced in a host cell in large quantity. Any polypeptide of interest can be expressed with the methods described herein. The term “poly-peptide” refers to a molecule comprising a polymer of amino acids linked together by a peptide bond(s). Polypeptides include polypeptides of any length, including proteins (e.g. having more than 50 amino acids) and peptides (e.g. 2-49 amino acids). Polypeptides include proteins and/or peptides of any activity, function or size, and include secreted proteins, a membrane-anchored protein or an intracellular protein.

Exemplary polypeptides and recombinant polypeptides include enzymes (e.g. proteases, kinases, phosphatases), receptors, transporters, bactericidal and/or endotoxin-binding proteins, structural polypeptides, membrane-bound polypeptides, glycoproteins, globular proteins, immune polypeptides, toxins, antibiotics, hormones, growth factors, blood fators, vaccines or othe like. The polypeptides can be peptide hormones, interleukins, tissue plasminogen activators, cytokines, immunoglobulines, including antibodies or functional antibody fragments or variants thereof and Fc-fusion proteins. The polypeptide of interest that is expressed as described herein may also be a subunit or domain of a polypeptide, such as a heavy chain or a light chain of an antibody, or a functional fragment or derivative thereof.

The terms “product of interest,” “polypeoide of interest,” “protein product,” “polypeptide product,” “recombinant protein,” “protein of interest,” and “recombinant polypeptide” are used interchangeably and can refer to such individual subunits or domains, or the final protein that is composed of the respective subunits or domains, depending on the context. In embodiments, the polypeptide of interest is an immunoglobulin molecule, suitably an antibody, or a subunit or domain thereof such as the heavy or light chain of an antibody. The term “antibody” as used herein refers to a protein comprising at least two heavy chains and two light chains connected by disulfide bonds. The term “antibody” includes naturally occurring antibodies as well as all recombinant forms of antibodies, e.g., humanized antibodies, fully human antibodies and chimeric antibodies. Each heavy chain is usually comprised of a heavy chain variable region (VH) and a heavy chain constant region (CH). Each light chain is usually comprised of a light chain variable region (VL) and a light chain constant region (CL). The term “antibody”, however, also includes other types of antibodies such as single domain antibodies, heavy chain antibodies, i.e. antibodies only composed of one or more, in particular two heavy chains, and nanobodies, i.e. antibodies only composed of a single monomeric variable domain. As discussed above, the polynucleotide encoding the polypeptide of interest may also encode one or more subunits or domains of an antibody, e.g. a heavy or a light chain or a functional fragment or derivative thereof, as polypeptide of interest. Such subunits or domains can be expressed either from the same or different expression cassettes. A “functional fragment or derivative” of an antibody in particular refers to a polypeptide which is derived from an antibody and is capable of binding to the same antigen in particular to the same epitope as the antibody. Examples of fragments or derivatives of an antibody include (i) Fab fragments, monovalent fragments consisting of the variable region and the first constant domain of each the heavy and the light chain; (ii) F(ab)2 fragments, bivalent fragments comprising two Fab fragments linked by a disulfide bridge at the hinge region; (iii) Fd fragments consisting of the variable region and the first constant domain CH1 of the heavy chain; (iv) Fv fragments consisting of the heavy chain and light chain variable region of a single arm of an antibody; (v) scF fragments, Fv fragments consisting of a single polypeptide chain, (vi) (Fv)2 fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular.

As described herein, in embodiments, the cell composes at least one heterologous polynucleotide encoding a selectable marker and/or a heterologous polynucleotide encoding a reporter polypeptide in addition to the heterologous polynucleotide encoding the product of interest. A “selectable marker” allows, under appropriate selective culture conditions, the selection of host cells expressing said selectable marker. Thereby, host cells successfully transfected with the expression vector can be selected under appropriate selection conditions. Typically, a selectable marker gene will confer resistance to a selection agent such as a drug, an antibiotic or other toxic agent, or compensate for a metabolic or catabolic defect in the host cell. It may be a positive or negative selection marker. In embodiments, the selection marker enables the host cell to survive and proliferate in the absence or reduction of a compound which is essential for survival and/or proliferation of the host cells lacking the selection marker. By cultivating the host cells in a medium which does not comprise the essential compound in a concentration high enough for survival and/or proliferation of the host cell or comprises a reduced amount of said essential compound, only host cells expressing the selection marker can survive and/or proliferate.

According to one embodiment, the selectable marker is a drug resistance marker encoding a protein that confers resistance to selection conditions involving the drug. A variety of selectable marker genes have been described (see, e.g., WO 92108796, WO 94/28143, WO2004/081 167 WO2009/080759, WO2010/097240). For example, at least one selectable marker may be used which confers resistance against one or more antibiotic agents. The selectable marker may according to one embodiment be an amplifiable selectable marker. An amplifiable selectable marker allows the selection of vector containing host cells and may promote gene amplification of said vector in the host cells. Selectable marker genes commonly used with eukaryotic cells, such as mammalian cells, include the genes for aminoglycoside phosphotransferase (APH), hygromycin phosphotransferse (hyg), dihydrofolate reductase (DHFR), thymidine kinase (tk), glutamine synthetase, asparagine synthetase, and genes encoding resistance to neomycin (G418), puromycin, hygromycin, zeocin, ouabain, blasticidin, histidinol D, bleomycin, phleomycin and mycophenolic acid.

A “reporter polypeptide” can be utilized and allows the identification of a cell expressing the reporter polypeptide based on the reporting characteristics (e.g. fluorescence). Reporter genes usually do not provide the host cells with a survival advantage. However, the expression of the reporter polypeptide can be used to differentiate between cells expressing the reporter polypeptide and those cells which do not. Therefore, a reporter gene enables the selection of successfully transfeeted host cells. Suitable reporter polypeptides include but are not limited to green fluorescent protein (GFP), yellow fluorescent protein (YFP), cyan fluorescent protein (CFP) and luciferase. As described, the expression vector comprising the polynucteotide encoding the product of interest may also comprise more than one selectable marker and/or reporter gene. Furthermore, the one or more polynucleotides encoding the selectable marker(s) and/or the one or more polynucleotides encoding the reporter polypeptide(s) may also be provided on one or more different expression vectors which are co-transfected with the expression vector which comprises the polynucleotide encoding the product of interest. Such co-transfection strategies likewise enable selection as is well-known in the art.

The expression vector or the combination of at least two expression vectors comprised in the cell may additionally comprise further vector elements. For example, at least one additional polynucleotide encoding a further product of interest can be utilized. The final polypeptide that is to be produced and suitably secreted by the host cell can also be a protein that is composed of several individual subunits or domains. An example of a respective protein is an immunoglobulin molecule, in particular an antibody that comprises heavy and light chains. There are several options for producing a respective protein that is composed of different individual subunits or domains and appropriate vector designs are known in the art. According to one embodiment, two or more subunits or domains of said protein are expressed from one expression cassette. In this embodiment, one long transcript is obtained from the respeetrye expression cassette that comprises the coding regions of the individual subunits or domains of the protein. According to one embodiment, at least one IRES element (internal ribosomal entry site) is functionally located between the coding regions of the individual subunits or domains and each coding region is preceded by a secretory leader sequence. Thereby, it is ensured that separate translation products are obtained from said transcript and that the final protein can be correctly assembled and secreted.

In some embodiments, such as the expression of antibodies, it is desirable to express the individual subunits or domains from different expression cassettes. According to one embodiment, the expression cassette used for expressing the product of interest is a monocistronic expression cassette. Expression cassettes comprised in the expression vector or combination of expression vectors may be monocistronic. According to one embodiment, each expression cassette designed for expressing a product of interest comprises a polynucleotide encoding one subunit or domain of the protein to be expressed as polypeptide of interest. In case of antibodies one expression cassette may encode the light chain of an antibody and another expression cassette may encode the heavy chain of the antibody. After expression of the individual subunits or domains from the individual expression cassettes, the final protein such as an antibody is assembled from said subunits or domains and secreted by the host cell. This embodiment is particularly suitable for expressing immunoglobulin molecules such as antibodies. In this case, a first heterologous polynucleotide encoding a product of interest encodes e.g. the heavy or the light chain of an immunoglobulin molecule and a second heterologous polynucleotide encoding a product of interest encodes the other chain of the immunoglobulin molecule.

### Methods of Producing Cells and Cell Lines

In additional embodiments, methods of producing an isolated cell for use in recombinant protein production are provided herein. The methods described herein suitably are used to produce a cell line which can be maintained and passaged, so as to allow for multiple experimental or protein production procedures.

In embodiments, the methods described herein comprise impairing an effect of an expression product of a ULK1 gene in the isolated cell and culturing the isolated cell under conditions that allow for expansion of the isolated cell.

Methods for impairing the effect of the expression product of a ULK1 gene (ULK protein) are described herein and include actions to impair the ULK1 gene as well as an expressed ULK1 protein.

In an exemplary embodiment, the methods include reducing or eliminating the functional expression of the ULK1 gene, thereby impairing the effect of the expression product of the ULK1 gene in the cell. Suitable ways are described herein. According to one embodiment, the genome of the cell (e.g., a eukaryotic cell) can be altered to reduce or eliminate the functional expression of the ULK1 gene. For example, a gene knock-out may be introduced into the ULK1 gene. According to one embodiment, such gene knock-out is introduced in all copies of the ULK1 gene. According to another embodiment, the ULK1 gene is deleted. All copies of the ULK1 gene may be deleted in the genome. According to one embodiment, the method comprises deleting a portion of a chromosome, wherein the deleted portion comprises the ULK1 gene. The deleted portion may correspond to a telomeric region. Such deletion can be induced e.g. by using an agent that induces chromosome breakages. Here, the cells can be repeatedly treated with such agent in order to obtain cells in which the functional expression of the ULK1 gene is reduced or eliminated, because all copies of said gene are deleted because of induced chromosome breaks.

Exemplary cells, including eukaryotic and suitable mammalian cells, are described herein, where the impairment of the ULK1 gene can be carried out. In suitably embodiments, the cells that are prepared are mammalian cells such as a Human Embryonic Kidney (HEK) cell or a Chinese Hamster Ovary (CHO) cell.

Methods of culturing the isolated cells and conditions under which the cells can be expanded are unique to the particular cell line, and are known in the art. Such culturing methods are disclosed in for example, Sambrook et al., Molecular Cloning: A Laboratory Manual. Cold Spring Harbor Laboratory Press, New York (1989). Culturing methods include the use of various culturing media and steps that allow for cell lines to be expanded to generate stable cells that can be stored and passaged, allowing for their continued use. Selection of cells with the appropriate impairment of the ULK1 gene can be carried out using the various methods described herein, including for the screening of the genetic makeup of the cell population.

As used herein “stable” or “stability” when referring to a cell line or cell culture, means that there is generally less than a 30% decrease in volumetric productivity (i.e., volumetric protein production (mg/mL)) over a period of at least 6-14 weeks of cell growth. This stability is a unexpected finding of the methods described herein that allow for the cell lines to be reproducibly used for increased protein production, even though the ULK1 gene or gene expression has been impaired in the cell line. In exemplary embodiments, the cell lines described herein exhibit a stability such that volumetric productivity decreases by less than 25%, less than 20%, less than 10%, or less than 5%, over a period of about 8-12 weeks, suitably about 8-10 weeks.

Cells may be cultured in an appropriate medium. An appropriate, or effective, medium refers to any medium in which a cell is capable of growing and/or expressing heterologous polypeptides/proteins of interest. Such a medium is typically an aqueous medium comprising carbon, nitrogen and phosphate sources, but can also include appropriate salts, minerals, metals and other nutrients. Microorganisms and other cells can be cultured in conventional bioreactors and by any process, including batch, fed-batch, cell recycle, and continuous fermentation. The pH of the culture medium is regulated to a pH suitable for growth and protein production of the particular organism. The growth chamber can be aerated in order to supply the oxygen necessary for growth and to avoid the excessive accumulation of carbon dioxide. Culture media and conditions for various host cells are known in the art.

Various methods for mutating or editing the ULK1 gene in the cells are described herein, and include nurtadon or knocking, out the ULK1 gene, as well as by post-transcriptional gene interference of the ULK1 gene.

As described herein, the impairing of the effect of the expression product of the ULK1 gene results in an increase in production of a recombinant protein as compared to a cell in which the effect of the expression product of the ULK1 gene has not been impaired. This increase in production is suitably on the order of at least about 30% more suitably about 50% to about 500%, relative to a cell in which the ULK1 gene/protein product that has not been impaired.

Exemplary recombinant proteins, including secreted proteins, membrane-anchored proteins and intracellular proteins, can be produced by the cells described herein.

### Methods of Producing a Product of Interest

In further embodiments, provided herein is a method of producing a product of interest, suitably a recombinant protein. Such methods include introducing a recombinant gene encoding the recombinant protein into an isolated cell. Suitably the cell has been modified as described herein, such that an effect of an expression product of a ULK1 gene has been impaired. The isolated cell is cultured under conditions that allow for expression of the recombinant protein. The recombinant protein is then isolated from the cell.

The cells, including eukaryotic cells, provided herein are suitable as production host cells for recombinantly producing a product of interest such as a poly peptide or protein of interest. Suitable examples of cells, wherein the effect of the expression product of the ULK1 gene in the cell is impaired. including by reducing or eliminating the functional expression of the ULK1 gene, as well as examples of the product of interest (recombinant proteins) are described herein in detail. As described herein, the product of interest, suitably a recombinant protein, is produced at a greater amount in the impaired cells than in those in which the ULK1 gene or the expression product of the gene, has not been impaired. As described herein, in embodiments, the amount of recombinant protein produced is increased by at least 30%, for example about 50% to about 500%, above that of a cell that does not containg an impaired ULK1 gene or ULK1 gene expression product.

As described herein, the eukaryotic cell suitably is a vertebrate cell, more suitably a mammalian cell. According to one embodiment, the methods comprises introducing into a eukaryotic cell a polynucleotide encoding a product of interest and selecting a host cell which expresses the product of interest. Introduction can be achieved by transfection as described above. Selection may occur using the methods described herein, including the use various reporter genes and selectable markers. Suitably, host cells are selected wherein the heterologous polynucleotide encoding the product of interest is stably integrated into the genome a the host cell. Exemplary host cells include Human Embryonic Kidney (HEK) cells and Chinese Hamster Ovary (CHO) cells, as well as other eukaryotic cells described herein.

Methods for isolating the product of interest, for example an expressed recombinant polypeptide or protein, are known in the art. Such methods include, for example, various cell lysis steps and filtrations, including the use of chromatomography separations, etc.

According to one embodiment, host cells are cultured under serum-free conditions. The expressed product of interest may be obtained by disrupting the host cells and then isolating the product. Suitably, the product of interest is a polypeptide or protein. The polypeptide is suitably expressed, e.g. secreted, into the culture medium and can be obtained therefrom. For this purpose, an appropriate leader peptide can provided in the polypeptide of interest. Leader sequences and expression cassette designs to achieve secretion are well known in the art. Thereby, polypeptides such as proteins can be produced and obtained/isolated efficiently with high yield.

The product of interest which suitably is a polypeptide of interest that is produced may be recovered, further purified, isolated, processed and/or modified by methods known in the art. For example, the polypeptide may be recovered from the nutrient medium by conventional procedures including, but not limited to, centrifugation, filtration, ultra-filtration, extraction or precipitation. Further processing steps such as purification steps may be performed by a variety of procedures known in the art including, but not limited to, chromatography (e.g. ion exchange, affinity, hydrophobic, chromatofocusing, and exclusion), electrophoretic procedures (e.g., preparative isoelectric focusing), differential solubility (e.g. ammonium sulfate precipitation) or extraction. Furthermore, the isolated and purified polypeptide of interest may be further processed and formulated, into a composition, e.g. a pharmaceutical composition.

The cell growth conditions for the cells described herein can include that which facilitates expression of the protein of interest. As used herein, the term “fermentation” includes embodiments in which literal fermentation is employed and embodiments in which other, non-fermentative culture modes are employed. In some embodiments, the fermentation medium may be selected from among rich media, minimal media, and mineral salts media.

An expression system as described herein can be cultured in any fermentation format. For example, batch, fed-batch, semi-continuous, and continuous fermentation modes may be employed herein. Wherein the protein is excreted into the extracellular medium, continuous fermentation is preferred.

Fermentation may be performed at any scale. Thus, microliter-scale, centiliter scale, and deciliter scale fermentation volumes may be used, and 1 Liter scale and larger fermentation volumes can be used. In some embodiments, the fermentation volume will be at or above 1 Liter. In another embodiment, the fermentation volume will be at or above 5 Liters, 10 Liters, 15 Liters, 20 Liters, 25 Liters, 50 Liters, 75 Liters, 100 Liters, 200 Liters, 500 Liters, 1,000 Liters, 2,000 Liters, 5,000 Liters, 10,000 Liters or 50,000 Liters.

Growth, culturing, and/or fermentation of a cell for producing a product of interest is performed within a temperature range permitting survival of the cells, preferably a temperature within the range of about 4° C. to about 55° C., inclusive. Thus, e.g., the terms “growth” (and “grow,” “growing”), “culturing” (and “culture”), and “fermentation” (and “ferment,” “fermenting”), as used herein in regard to the cells, inherently means “growth,” “culturing,” and “fermentation,” within a temperature range of about 4° C. to about 55° C., inclusive. In addition, “growth” is used to indicate both biological states of active cell division and/or enlargement, as well as biological states in which a non-dividing and/or non-enlarging cell is being metabolically sustained, the latter use of the term “growth” being synonymous with the term “maintenance.”

Expression of a product of interest may lead to production of extracellular polvoeptides or proteins. The methods may also include the step of purifying the polypeptides of proteins of interest from the periplasm or from extracellular media. In some embodiments, the methods provided herein allow for production of a product of interest, e.g., a protein, and then recovering the protein from the cell culture. In some embodiments, recovering the protein comprises centrifugation to remove cells and/or cellular debris. In some embodiments, recovering the protein comprises filtering to remove cells and/or cellular debris.

Products of interest, in particular proteins and peptides can be isolated and purified to substantial purity by standard techniques well known in the art, including, but not limited to, ammonium sulfate or ethanol precipitation, acid extraction, anion or cation exchange chromatography, phosphocellulose chromatography, hydrophobic interaction chromatography, affinity chromatography, nickel chromatography, hydroxy apatite chromatography, reverse phase chromatography, lectin chromatography, preparative electrophoresis, detergent solubilization, selective precipitation with such substances as column chromatography, immunopurification methods, and others. For example, proteins having established molecular adhesion properties can be reversibly fused with a ligand. With the appropriate ligand, the protein can be selectively adsorbed to a purification column and then freed from the column in a relatively pure form. The fused protein is then removed by enzymatic activity. In addition, protein can be purified using immunoaffinity columns or Ni-NTA columns. General techniques are fUrther described in, for example, R. Scopes, Protein Purification: Principles and Practice, Springer-Verlag: N.Y. (1982); Deutscher, Guide to Protein Purification, Academic Press (1990), U.S. Pat. No. 4,511,503; S. Roe, Protein Purification Techniques: A Practical Approach (Practical Approach Series), Oxford Press (2001); D. Bollag, of al., Protein Methods, Wiley-Lisa, Inc. (1996); A K Patra et al, Protein Expr Purif, 18(2): p/182-92 (2000); R. Mukhija, et al., Gene 165(2): p. 303-6 (1995). See also, for example, Ansubel, et al. (1987 and periodic supplements); Deutscher (1990) “Guide to Protein Purification,” Methods in Enzymology vol. 182, and other volumes in this series; Coligan, et al. (1996 and periodic Supplements) Current Protocols in Protein Science Wiley/Greene, NY; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protean removable sequence. See also, for example, Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, NY; and Crowe, et al. (1992) QIAexpress: The High Level Expression & Protein Purification System QUIAGEN, Inc. Chatsworth, Calif.

Detection of an expressed protein can also be achieved by methods known in the art and include, for example, radioimmunoassays, Western blotting techniques or immunopreciptation.

Expressed recombinant proteins and polypeptides present in the supernatant of a cell can be separated from the host proteins by standard separation techniques well known to those of skill in the art. For example, an initial salt fractionation can separate many of the unwanted host cell proteins (or proteins derived from the cell culture media) from the heterologous polypeptide of interest. One such example can be ammonium sulfate. Ammonium sulfate precipitates proteins by effectively reducing the amount of water in the protein mixture. Proteins than precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of a polypeptide or protein of interest can be used to isolate it from proteins greater and lesser size using ultrafiltration through membranes of different pore size (for example, Amicon or Millipore membranes). As a first step, the protein mixture can be ultrafiltered through a membrane with a pore size that has a lower molecular weight cut-off than the molecular weight of the protein of interest. The retentate of the ultrafiltration can then be ultrafiltered against a membrane with a molecular cut off greater than the molecular weight of the protein of interest. The heterologous polypeptide of interest will pass through the membrane into the filtrate. The filtrate can then be chromatographed as described below.

The polypeptide of interest can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Methods for large scale production of products of interest, particularly recombinant polypeptides and proteins, are known in the art and include the use of cell cultures or at procedures. Examples of such large scale production include batch, fed-batch, cell recycle, and continuous fermentation.

### Methods for Screening for Genes that Result in Increased Protein Purification

In additional embodiments, provided herein are methods of screening for a gene, an expression product of which when impaired, results in increased production of a recombinant protein. The methods described herein can be used to detect, determine, or confirm that a gene may be involved m protein production by a cell, and if impaired, or the expression product thereof is impaired, allows for increased protein production by the cell.

The methods include impairing a function of the expression product of the gene in an isolated cell to create an impaired isolated cell. Various methods of impairing the function of a gene and its expression product are described herein and include gene mutation, editing and gene knock-out, various post-transcriptional gene impairment methods such as RNA interference, as well as impairment of the expression product itself by directly impairing the function or action of the expression product of the desired gene.

The methods further include introducing a recombinant gene (i.e., nucleic acid) encoding the recombinant protein into the impaired isolated cell. Various methods of introducing a recombinant gene, including vectors, etc., are described herein.

The impaired cell is then cultured under conditions that allow for expression of the recombinant protein. Exemplary conditions are described herein and also well known in the art.

The recombinant protein, if produced, is then isolated from the cell. Various methods for isolating recombinant proteins, including filtration methods, are known in the art and described herein. The volume of the recovered, isolated recombinant protein is then determined and compared between the impaired isolated cell, with the volume of production of the recombinant protein in a cell in which the effect of the expression product of the gene has not been impaired. That is, the amount of recombinant protein is determined in both impaired and control cells and compared in order to determine if impairing the gene (or the expression product of the gene) results in increased protein production. An increase in the volume of production of the recombinant protein in the impaired isolated cell of at least about 30% is indicative that the gene, the expression product of which, when impaired, results in increased production of recombinant protein. Suitably, the increase in the volume production is about 50% to about 500%.

As described herein, the cells that can be utilized in screening for such genes are suitably eukaryotic cells, including mammalian cells such as HEK and CHO cells.

Various methods for impairing the expression product of the gene are described herein and include mutating, editing or knocking out a gene. Methods for impairing the function or expression of the expression product of the gene (such as various post-transcriptional modifications) can be used to determine if a gene is involved in protein production in a cell.

Genes involved in mechanisms such as gene transcription, protein translation, metabolism of proteins, secretory capacity of cells, and apoptosis, are suitable classes of genes that can be screened in order to determine their effect on protein production, if impaired. A high-throughput assay as described herein allows for the discovery of a large number or potential genes (and expression products that can be targeted.

### EXAMPLES

### Example 1: Development of Screening Method for Genes Related to Recombinant Protein Expression

The following methods were developed to screen for endogenous genes, which if impaired, would improve recombinant protein expression. As described below, a high-throughput screening technology was developed that enabled determination of proteins (and thus genes) that regulate recombinant protein expression.

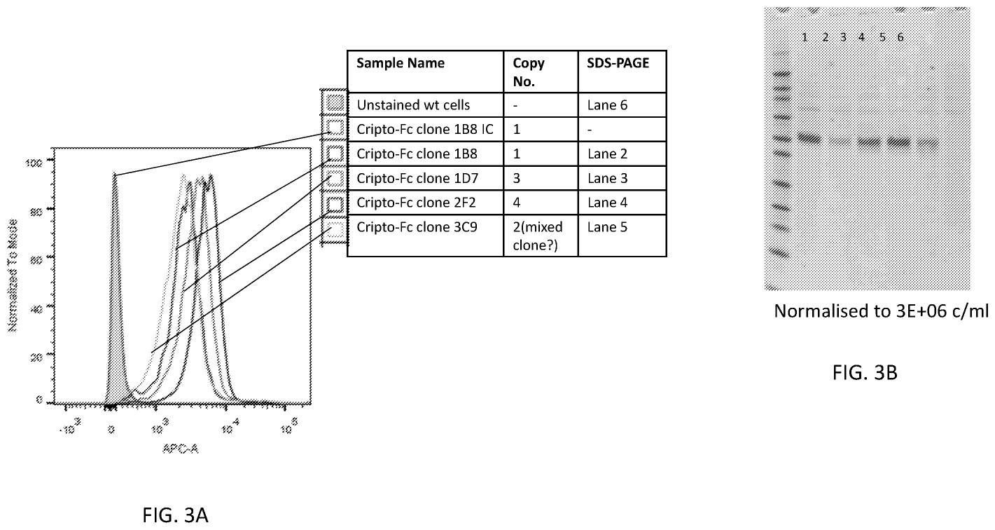

Human Embryonic Kidney (HEK) cells, Expi293F cell line, were co-transfected using Expifectamine with vectors for targeted integration of one of three model proteins, Cripto-Fc, PLAP and hGFRα2, and a vector expressing Zinc Finger Nucelases that target the AAVS1 locus in the genome. As shown below in Table 3, Cripto-Fc served as a high volume expressing protein, PLAP a medium expressing protein, and hGFRα2 as a low expressing protein. After 48 hours, selection with puromycin blasticidin were applied to enrich for cells with the vectors integrated. After 14 days, cells were sorted by flow cytometry as single cells in 96 well plates. After single colonies were isolated the cells were expanded and analyzed to determine the gene copy number of the recombinant target gene in each clone. The analysis for gene copy number was performed using digital droplet PCR comparing the copy number of the recombinant gene to a known reference gene (XX). For each of the model proteins two cell lines were identified that contain one and three (SEAP) or four (GFRa2 and CriptoFC) integrated gene copies, respectively. The expression of the recombinant proteins were confirmed and found to correlate with gene copy number as shown for CriptoFC in FIGS. 3A and B. For all screening experiments, cell lines containing a single copy of the integrated vector were used, and the cell lines with multiple gene copies were used as positive controls for increased protein expression.

TABLE 3
Model Proteins

| | Mw | Glyco- | Disul- | Ab | Expression |
|---|---|---|---|---|---|
| Protein | (kDa) | sylation | phides | Available | Level (mg/L) |
| Cripto-Fc | 46.6 | 1 (N) | 3 | Y | 1000 |
| (P13385) | | | | | |
| PLAP | 56.9 | 2 (N) | 2 | (Y) | 100 |
| (P05187) | | | | | |
| GFRα2 | 51.5 | 3 (N) | 0 | Y | 10 |
| (O00451) | | | | | |

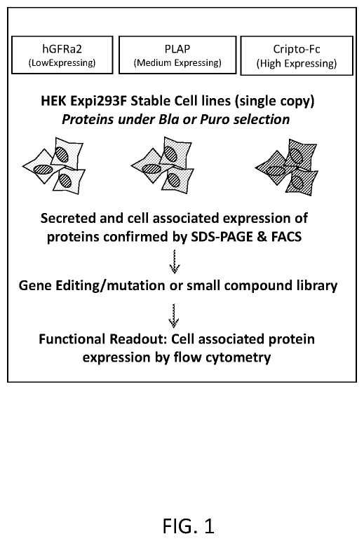

Once expression clones for each protein model were selected, gene editing/mutating or expression protein impairment (e.g., via small molecule impairment), could be carried out to determine the effect that different genes have on recombinant protein expression. For each of the model proteins, two cell lines were isolated that contain a single or multiple gene copies respectively. FIG. 1 shows a flowchart of the experimental design for screening for genes of interest.

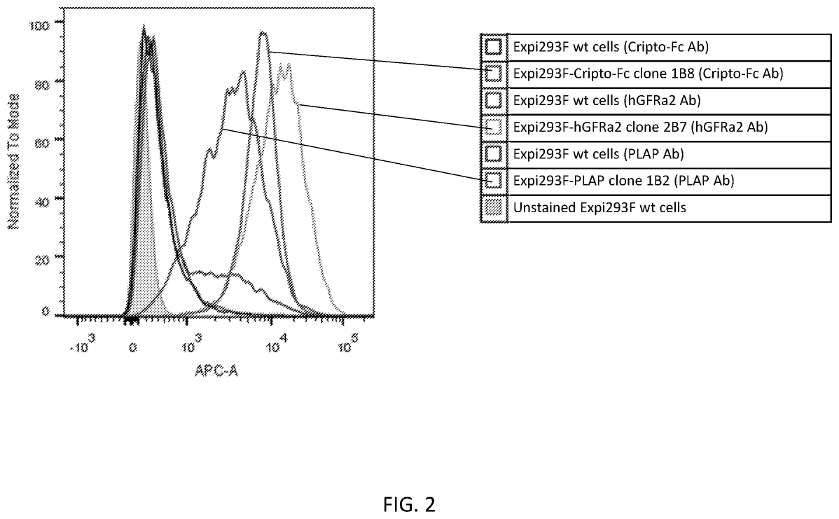

FIG. 2 shows a flow cytometry analysis (FACS) of Expi293F cells, expressing the different model proteins. As noted, antibodies for the model proteins showed the expression of each of the proteins in the cells, verifying that the model system could be used to assess the effects of impaired gene function on recombinant protein expression. Wildtype cells assayed with antibodies against the model proteins did not show expression of the proteins, except for PLAP were some background expression of endogenous proteins was detected. FIG. 3A illustrates that the developed FACS methods can differentiate between different levels of protein expression (Cripto-Fc), examining the impact of delivery of different copy numbers (1, 3, 4). FIG. 3B confirms the expression levels of Cripto-Fc via SDS-Page. These results illustrate that the model system can be used to investigate the effects of impairing gene function and or expression product function of various genes to determine their effects on recombinant protein expression. In addition, the model has been extended to a 384 well system, enabling high throughput experiments and analysis.

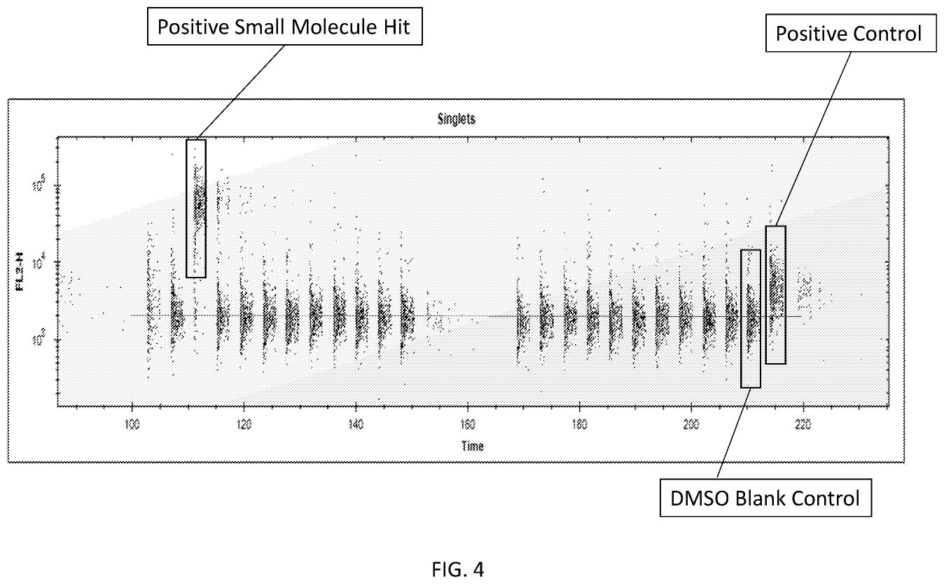

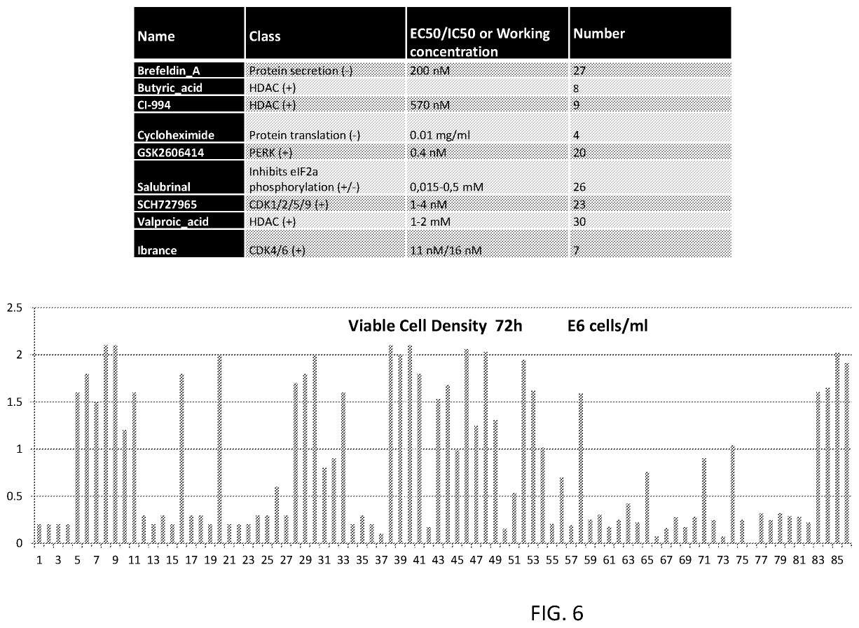

FIG. 4 shows screening data across 24 wells in a primary screening. The X-axis displays time of sampling from each well. Each data point corresponds to the flourescence (Y-axis) of a single cell in the sampled well. DMSO (blank) control of the cell line containing a single gene copy of the CriptoFC gene is shown in FIG. 6. Also shown is the positive control which is the CriptoFC cell line containing 4 copies of the CriptoFC gene. The sample appearing between 110-115 on the X axis is the result from a well containing positive small molecule hit.

### Example 2: Screening of Small Molecule Compounds to Provide Increased Protein Expression

The cell assay described above was utilized to screen a large library of small molecule compounds for the ability to increase recombinant protein expression. Approximately 19,2000 compounds were tested. The library was composed of compounds on which information on their biological activity is available. Each of the compounds has a biological activity on at least one protein target with an EC₅₀<=100 nM. The HEK293 CriptoFC 1B8 cell line was used for primary screening. Cells were plated in 384 well plates and treated with 1.2 uM of compound for 72 hours. The cells were then stained with antibodies towards CriptoFC conjugated to a fluorescent label to enable flourescent detection. Expression of CriptoFC were measured by flow cytomtery on live, single cells as shown in FIG. 4. Using a cut-off at Z-score>10 there were 515 compounds identified as having a positive effect on recombinant protein expression in the primary screen.

The Z Score calculation calculates the center and spread of the data on a plate. In an HTS plate, the median activity of compound wells is centered at zero with a spread measured around the zero defined by a robust standard deviation. Each individual compound is then measured in units of robust standard deviation of how far its activity is from the median of the plate using the following equation:

$$Robust z score = \frac{x - m}{RSD}$$

Where x=the well raw data value, m=median of chosen contorl well group and RSD is the robust standard deviation (MAD*1.483) for the chosen well group.

A Z Score of 3 indicates that a compound's activity is 3 rSD from the median of the plate and is a strong indication of an active compound (3SD=99.7% confidence). A key benefit Z Score is that is it compares compound activities between plates and assays, taking into account the spread (or noise) of the data which a calculation of % activity does not.

To confirm hits from the primary screen, the available positive hits were subjected to the counter screening without addition of antibody-conjugates to remove flourescent compounds. In parallel, a 10 point concentration response curve with a top concentration of 6 μM for each of the compounds was determined. Only compounds that lacked inherent fluorescence, and increased protein expression with at least 30% in a dose-dependent mariner were counted as confirmed hits. In total, 98 compounds were confirmed as true hits that increased CriptoFC expression in the HEK293 CriptoFC 1B8 cell line. Using available knowledge about the biological activity of the compounds, the following list of genes in Table 4 were determined to be possible targets for impairment and resultant increase recombinant protein production.

TABLE 4
Target Gene list based on
Cripto-FC screen
ATM
BRD4
CHEK1
EGFR
ESR1
JAK2
JAK3
KIAA1804
LCK
MAP3K7
PAK1
PDPK1
PRKCQ
RET
ULK1
TYMS
EHMT2
BIRC2
KIAA1551
GMNN
PGR
NFE2L2
AR

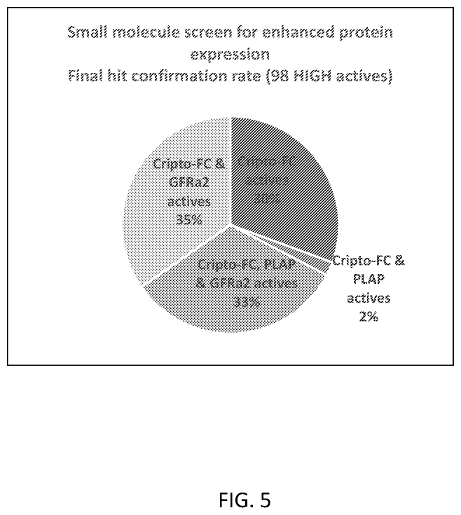

An additional study was performed on different cells expressing GRFα2 and PLAP. The same assay performed on HEk293 CriptoFC 1B8 was performed on a cell line containing a single gene copy of GFRA2. Since there was a slightly increased background of endogenous PLAP in the HEK293 cells (FIG. 2) the expression of PLAP was determined by measuring the enzymatic activity secreted by the HEK293 PLAP cell line. The activity measurement for PLAP is based on the degradation of p-Nitrophenyl phosphate to p-Nitrophenyl which can be followed as an increase in absorbance at 405 nm. FIG. 5 shows the outcome of confirmation screening of the 98 primary hits that were active on the CriptoFC cell line on the cell lines expressing PLAP and GFRa2. 32 (33%) of the 98 hits had a significant positive effect of the specific productivity of all three model proteins, while 37% (35±2) only affected two model proteins, and finally 30% only had a positive effective on the ITEK 293 CriptoFC 1B8 cell line.

### Example 3: Determination of ULK1 as a Target Gene of Interest

The following Example describes the experiments that sere undertaken in determining ULK1 as a gene, the impairment of which, results in increased recombinant protein production.

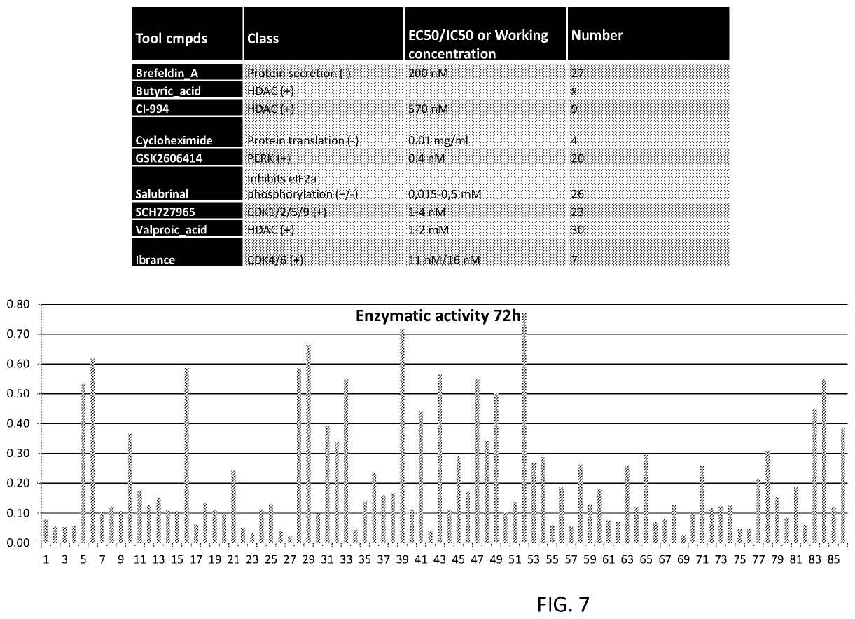

A study was conducted in 5 ml cultures with the HEK293 PLAP cell line to compare with compounds suggested in the literature to be involved in recombinant protein expression. The cells were seeded at 0.2×10⁵ cells/ml and treated with compounds for 72 hours. Each compound was used at 10 times the determind EC₅₀, or a maximum of 5 μM. After 72 hours, the viable cell density (FIG. 6) and MAP activity (FIG. 7) were determined. Analyzing the annotation of biological activity for the 10 compounds in this experiment which had the least impact on cell viability and growth properties, concomitant with the highest positive effect on PLAP expression, ULK1 was singled out as the top candidate gene responsible for the phenotypic effect (TABLE 6).

FIG. 6 shows the effect of the compounds on cell viability, measured as viable cell density at 72 hours. FIG. 7 shows the enzymatic activity of the PLAP gene product, measured in these same cells at 72 hours. Based on these studies, compounds having the greatest effect on enzymatic activity (highest protein production), while not impacting cell viability, were selected for further analysis.

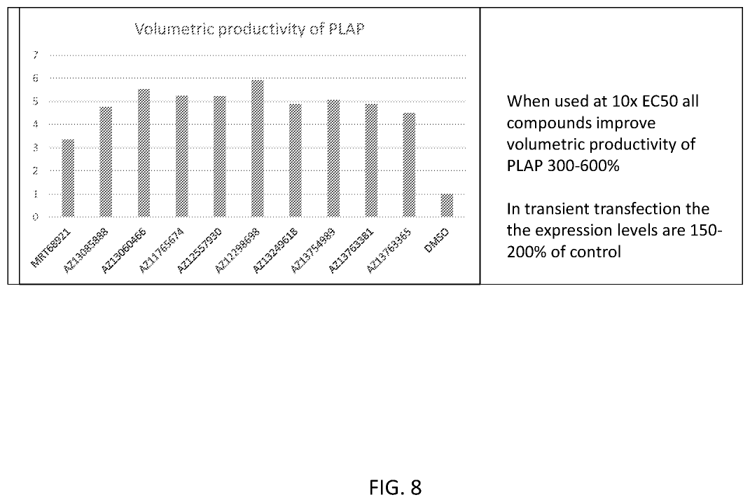

FIG. 8 shows the volume productivity of PLAP of select compounds. Table 5 below shows the AC50 concentration of each of the compounds on three different protein targets, including the compound MRT68921. As indicated in FIG. 8, all compounds increased volumetric productivity of PLAP by about 300-600%.

TABLE 5
pAC50 for Improved Protein Expression using Three Different Protein Targets

| | AZ12298698 | AZ12557930 | AZ13763365 | AZ13754989 | AZ13060466 | AZ13763381 |
|---|---|---|---|---|---|---|
| Cripto-FC | 7.11 | 7.17 | 6.05 | 6.9 | 7.67 | 6.45 |
| PLAP | 7.41 | 7.22 | 6.55 | 6.97 | 4.5 | 6.49 |
| GFRa2 | 7.44 | 6.93 | 6.13 | 7.15 | 7.43 | 6.44 |

| | | | |
|---|---|---|---|
| AZ13085888 | AZ11765674 | AZ13249618 | MRT68921 |

| | | | | |
|---|---|---|---|---|
| Cripto-FC | 6.73 | 6.9 | 6.38 | 7.10 |
| PLAP | 6.6 | 6.65 | — | 7.22 |
| GFRa2 | — | 6.63 | 6.41 | — |

An analysis of the best hits from this study revealed that 8/10 hits were directed to the ULK1 gene expression product. Table 6 below shows the breakdown of targets.

TABLE 6
Target Breakdown from Phenotypic Screen
Compounds that increase
recombinant protein ex-
pression without effects on
cell viability and growth

| | |
|---|---|
| AZ13763365 | ULK1 |
| AZ12298698 | ULK1 |
| AZ12557930 | ULK1 |
| AZ13754989 | ULK1 |
| AZ13060466 | ULK1 |
| AZ13763381 | ULK1 |
| AZ12064463 | ADCYC |
| AZ13085888 | ULK1 |
| AZ11765674 | Kinase |
| AZ13249618 | ULK1 |

To further explore how impairing the activity of the expression product of the ULK1 gene impacts protein production, studies were carried out to knock out or edit the ULK1 gene using CRISPR. The ULK1 gene was modified in HEK293 CriptoFC cells, and Cripto-Fc was used as the recombinant protein product, as discussed above. For the knock out experiments, HEK293 CriptoFC 1B8 cells were co-transfected with vectors encoding Cas9 and guide RNA at 1:1 ratio. For the ULK1 knock out experiment, two guide RNA (SEQ ID NOS:3 and 4 below) were simulaneously co-transfected with Cas9.

ULK1 gRNA C8
(SEQ ID NO: 3)
ACCGCGGCGGCACAGAGACCG for human ULK1
(281-299 in exon 1)
ULK1 gRNA D8
(SEQ ID NO: 4)
ACCGCCACGGCGCCTTCGCGG for human ULK1
(336-353 in exon 1)

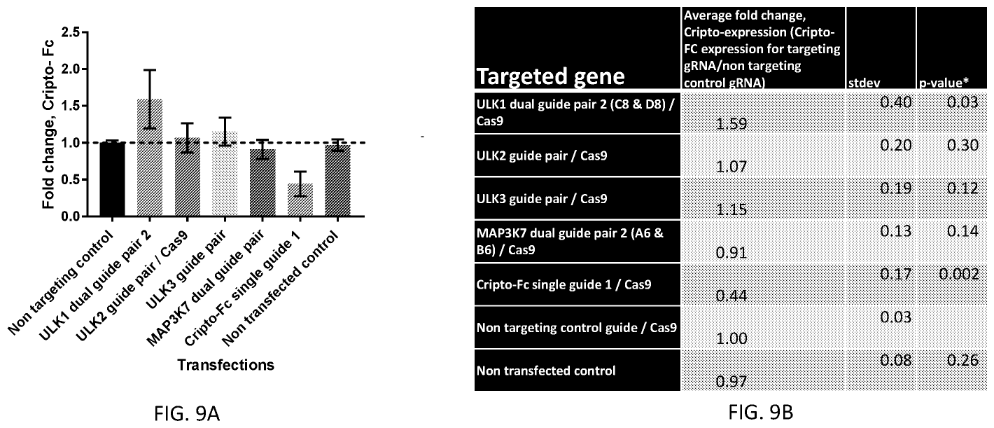

The vector encoding Cas9 also express green fluorescent protein which enabled an enrichment of fluorescent cells 2 days after transfection. The analysis of protein expression was performed 6 days after transfection by flow cytometry as described above. The results are shown in FIGS. 9A and 9B. As illustrated, CRISPR-Cas9 gene editing performed on the ULK1 gene resulted in a 1.6-fold increase in Cripto-Fc enzymatic activity, illustrating increased protein production, relative to non-targeted approaches. No significant changes were noted when ULK2, ULK3 or MAP3k7 genes were edited using CRISPR-Cas9 gene editing.

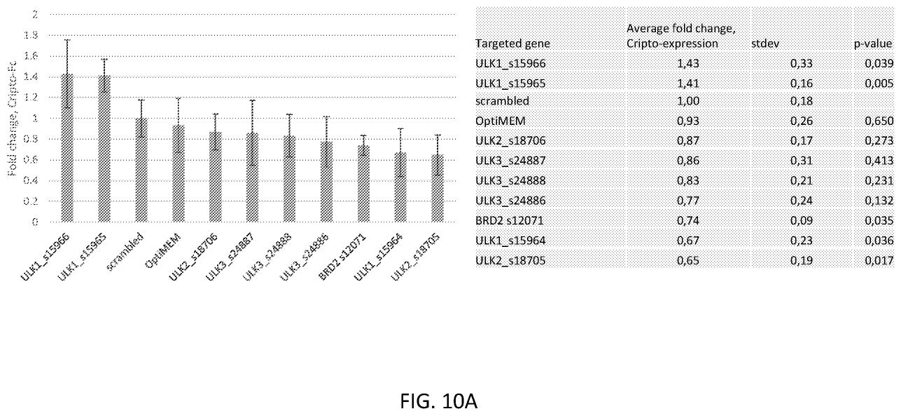

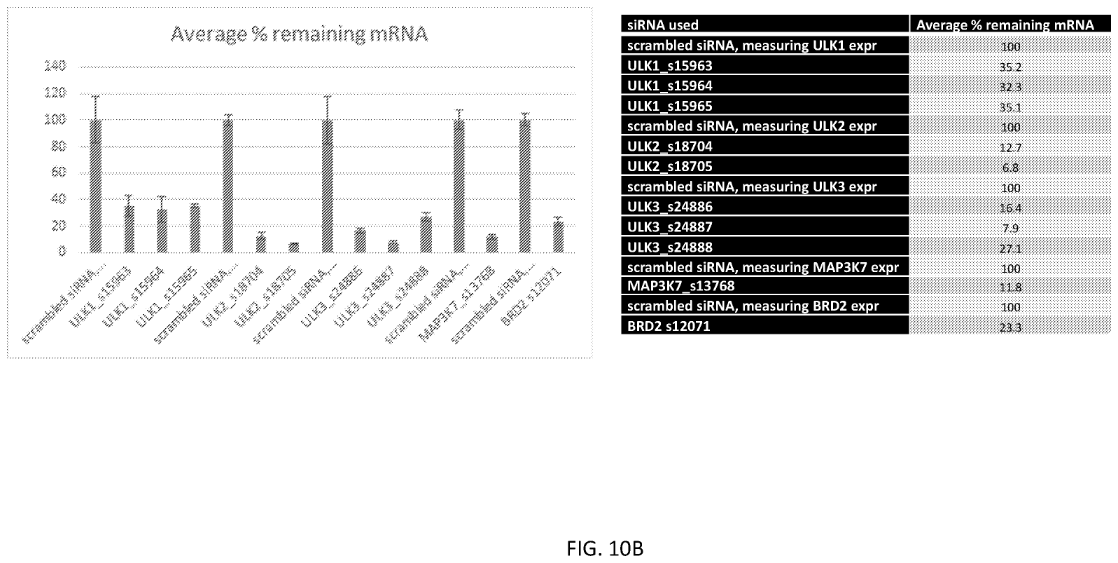

siRNA was also used to down-regulate the ULK1 gene in Expi293-Cripto-Fc cells. As shown in FIG. 10A, siRNA directed against the ULK1 gene resulted in a 1.4 fold increase in protein production, relative to a scrambled control. No other targeted gene provided any increase in protein production. Experimentally, HEK293 CriptoFC 1B8 cells were seeded at 1230 cells/well in a 384 well plate and transfected with 30 nM siRNA using Expifectamine to make transfection complexes. The plates were incubated at 37° C. for three days before CriptoFC expression was analyzed as described above. FIG. 10B shows the extent of mRNA knock down (average % remaining) achieved using the various siRNA constructs, demonstrating the effect of reducing mRNA expression.

### Example 4: Demonstration of Increased Recombinant Protein Production in Cell Lines with Impaired ULK1 Activity

After determination that ULK1 was responsible for improved capability of recombinant protein production as demonstrated above, two clonal HEK293 cell lines in which the ULK1 gene was impaired were isolated. For the cell line generation, unmodified HEK2.93 and HEK293 CriptoFC 1B8 cells were co-transfected with vectors encoding Cas9-T2A-GFP and guide RNA at 1:1 ratio. For the knock out experiment, two guide RNA (SEQ ID NOS:5 and 6 below) were simultaneously co-transfected with Cas9-T2A-GFP (ratio 1:0.5:0.5 for Cas9:gRNA 1a:gRNA 1b).

ULK1 gRNA 1a
(SEQ ID NO: 5)
AGACCAAAGCGAAGGCGCCG for human ULK1
ULK1 gRNA 1b
(SEQ ID NO: 6)
CGGCCCGGGATCCCCCGCCC for human ULK1

Clonal cell lines were obtained by fluorescence assisted cell sorting of simile GFP positive cells into 96 well plates. The cells were maintained in DMEM+10% fetal bovine serum, 37° C. and 5% CO₂. Once sufficient cells had been obtained of each clone they were adapted back into suspension culture and Expi293 expression medium. Genetic analyses through PCR across the intended deletion site allowed identification of clones that the desired deletion across ULK1 gene alleles. For each cell background one clone was selected for further expenment, Expi293 ULK KO (9F6) and Expi293 1B8 ULK1 KO (1C11).

Fragment analysis of
Expi293 ULK1 KO clones

| | Fragment |
|---|---|
| Clone | size |
| 9G7 | 542, 481 |
| 9E9 | 508/495 |
| 10B6 | 511, 502, 487, 443 |
| 10G11 | 511 |
| 11B5 | 511 |
| 11C7 | 457, 445, 443 |
| 11 E7 | 482, 444, 437 |
| 9C4 | 509 |
| 9F6 | 443 |
| 11E2 | 438, 474 |

Fragment analysis of Expi293-
1B8 ULK1 KO clones

| Clone | Fragment |
|---|---|
| 188 | size |
| 1EZ | 479, 492, 495 |
| 1C11 | 442 |
| 2G2 | 510 |
| 2B11 | 478, 502 |
| 3G3 | 510 |
| 3O7 | 473, 479, 482 |
| 4C8 | 487 |
| 3B9* | 488, 497 |

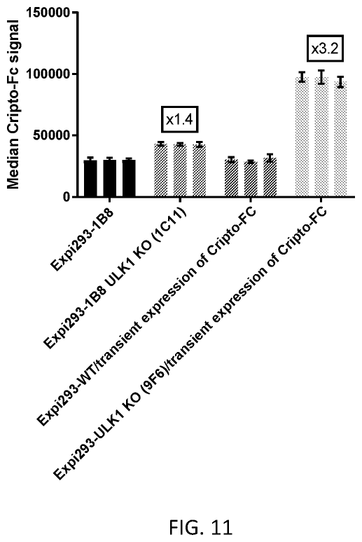

The Expi293 and Expi293 ULK1 KO (9F6) were transiently transfected with the plasmid encoding Cripto-FC that was used for the generation of the HEK293 CriptoFC 1B8 cell line. Cells were seeded 2×10⁶ cells/ml in Expi293 expression media the day before transfection and incubated at 37° C. and 8% CO₂, 150 rpm in Erlenmeyer flasks. The following day the cells were transfected with 1 microgram/ml plasmid DNA in complex with polyethylene imine. For six days the cells were incubated at 37° C. and 8% CO₂, 250 rpm in tubesin 50 tubes before protein expression levels were assessed. The cell lines with stable expression of Cripto-FC were seeded at 0.5×10⁶ cell/ml and incubated for three days under identical conditions before the CriptoFC expression levels were measured. As can be seen in FIG. 11 the cell lines in which the ULK1 gene had been knocked out the recombinant CriptoFC expression is increased in boat stable (1.4x) and transiently (3.2x) transfected cell lines.

A Chinese hamster ovary (CHO) cell line expressing human IgG1 Ab001 was generated by transfecting CHO suspension cells with an expression plasmid using an Amaxa nucleofection system and reagents (Lonza). The expression plasmid encoded the Ab001 heavy and light chain genes in addition to a glutamine synthetase selectable marker. Transfected cells were selected and maintained in proprietary medium in the presence of 50 μM methionine sulphoximine (Sigma-Aldrich). A clonal cell line was derived by limiting dilution cloning and routinely cultured at 37° C. in 5% (v/v) CO₂ in vented Erlenmeyer flasks (Corning), shaking at 140 rpm, and subcultured every 3-4 days. Cell concentration and viability were determined by an automated Trypan Blue exclusion assay using a Vi-Cell cell viability analyser (Beckman-Coulter).

A CHO cell line expressing Ab001 was inoculated at 7×10⁵ cells/ml in a final volume of 45 mL in replicate flasks. The ULK1 inhibitor was diluted in water. On day 3 post inoculation, an aliquot of ULK1 inhibitor was added to triplicate cultures to a final concentration of 1 μM and compared to triplicate imtreated cultures. Over the culture period, all the cultures were supplemented with five bolus additions of a proprietary nutrient feed and the levels of glucose were monitored and maintained. Culture samples were collected over the time course and were used to quantify Ab001 titre using Protein A analysis.

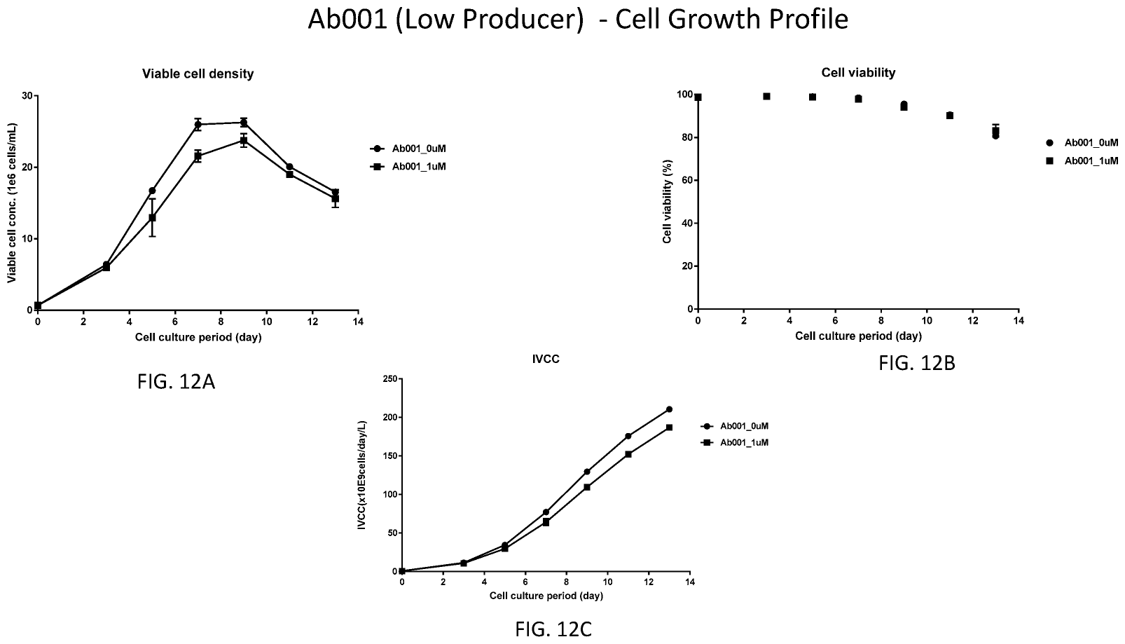

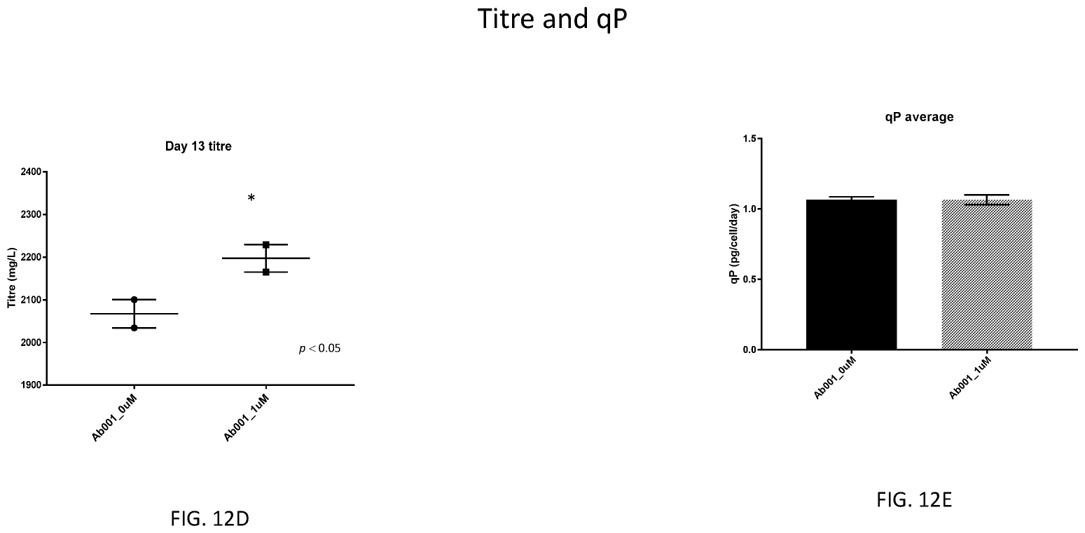

A single dose of 1 μM ULK1 inhibitor was sufficient to achieve a significant increase in volumetric litre in a stable cell line expressing Ab001, with minimal impact on cell growth and cell viability (FIGS. 12A-B).

It will be readily apparent to one of ordinary skill in the relevant arts that other suitable modifications and adaptations to the methods and applications described herein can be made without departing from the scope of any of the embodiments. The following examples are included herewith for purposes of illustration only and are not intended to be limiting.

It is to be understood that while certain embodiments have been illustrated and described herein the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scop of the present technology. Thus, the breadth and scope of the present technology should not be limited by any of the above-described embodiments, but should be defined only in accordance with the appended claims and their equivalents. It will also he understood that each feature of each embodiment discussed herein, and of each reference cited herein, can be used in combination with the features of any other embodiment. All patents and publications discussed herein are incorporated by reference herein in their entirety.

1. An isolated cell in which an effect of an expression product of a ULK1 gene has been impaired.

2. The isolated cell of claim 1, wherein the isolated cell is a eukaryotic cell.

3. The isolated cell of claim 2, wherein the eukaryotic cell is a mammalian cell.

4. The isolated cell of claim 3, wherein the mammalian cell is a Human Embryonic Kidney (HEK) cell or a Chinese Hamster Ovary (CHO) cell.

5. The isolated cell of claim 1, wherein the effect of the expression product of the ULK1 gene has been impaired by mutating or editing the ULK1 gene in the isolated cell.

6. The isolated cell of claim 5, wherein the mutation or edit eliminates or impairs one or more catalytic residues of the expression product of the ULK1 gene, or one or more residues of the expression product of the ULK1 gene that are post translationally modified, or results in a dominant negative mutation.

7. The isolated cell of claim 5, wherein the mutation or edit reduces the expression of, or knocks out, the ULK1 gene in the cell.

8. The isolated cell of claim 1, wherein the effect of the expression product of the ULK1 gene has been impaired by post-transcriptional gene interference.

9. The isolated cell of claim 8, wherein the post-transcriptional gene interference utilizes siRNA interference, microRNA interference, antisense RNA interference, or small molecule interference.

10. The isolated cell of claim 1, wherein the impairment of the effect of the expression product of the ULK1 gene results in an increase in production of a recombinant protein as compared to a cell in which the effect of the expression product of the ULK1 gene has not been impaired.

11. The isolated cell of claim 10, wherein the recombinant protein is a secreted protein, a membrane-anchored protein, or an intracellular protein.

12. The isolated cell of any of claim 11, wherein the recombinant protein is an antibody.

13. The isolated cell of any of claim 11, wherein the increase in production of the recombinant protein is at least about 30%.

14. The isolated cell of claim 13, wherein the increase in production of the recombinant protein is about 50% to about 500%.

15. A method of producing a recombinant protein, comprising:
a. introducing a recombinant gene encoding the recombinant protein into an isolated cell in which an effect of an expression product of a ULK1 gene has been impaired;
b. culturing the isolated cell under conditions that allow for expression of the recombinant protein; and
c. isolating the recombinant protein from the isolated cell of from the culture medium if the recombinant protein is secreted by the isolated cell.

16. The method of claim 15, wherein the isolated cell is a eukaryotic cell.

17. The method of claim 16, wherein the eukaryotic cell is a mammalian cell.

18. The method of claim 17, wherein the mammalian cell is a Human Embryonic Kidney (HEK) cell or a Chinese Hamster Ovary (CHO) cell.

19. The method of claim 15, wherein the effect of the expression product of the ULK1 gene has been impaired by mutating or editing the ULK1 gene in the isolated cell.

20. The method of claim 19, wherein the mutation or edit eliminates or impairs one or more catalytic residues of the expression product of the ULK1 gene, or one or more residues of the expression product of the ULK1 gene that are post translationally modified, or results in a dominant negative mutation.

21. The method of claim 19, wherein the mutation or edit reduces the expression of, or knocks out, the ULK1 gene in the cell.

22. The method of claim 15, wherein the effect of the expression product of the ULK1 gene has been impaired by post-transcriptional gene interference.

23. The method of claim 22, wherein the post-transcriptional gene interference utilizes siRNA interference, microRNA interference, antisense RNA interference, or small molecule interference.

24. The method of claim 15, wherein the impairment of the effect of the expression product of the ULK1 gene results in an increase in production of the recombinant protein as compared to a cell in which the effect of the expression product of the ULK1 gene has not been impaired.

25. The isolated cell of claim 24, wherein the recombinant protein is a secreted protein, a membrane-anchored protein, or an intracellular protein.

26. The method of claim 24, wherein the recombinant protein is an antibody.

27. The method of claim 24, wherein the increase in production of the recombinant protein is at least about 30%.

28. The method of claim 27, wherein the increase in production of the recombinant protein is about 50% to about 500%.

29. A method of producing an isolated cell for use in recombinant protein production, comprising:
a. impairing an effect of an expression product of a ULK1 gene in the isolated cell; and
b. culturing the isolated cell under conditions that allow for expansion of the isolated cell.

30. The method of claim 29 wherein the isolated cell is a eukaryotic cell.

31. The method of claim 30, wherein the eukaryotic cell is a mammalian cell.

32. The method of claim 31, wherein the mammalian cell is a Human Embryonic Kidney (HEK) cell or a Chinese Hamster Ovary (CHO) cell.

33. The method of claim 29, wherein the effect of the expression product of the ULK1 gene is impaired by mutating or editing the ULK1 gene in the isolated cell.

34. The method of claim 33, wherein the mutating or editing eliminates or impairs one or more catalytic residues of the expression product of the ULK1 gene, or one or more residues of the expression product of the ULK1 gene that are post translationally modified, or results in a dominant negative mutation.

35. The method of claim 33, wherein the mutating or editing reduces the expression of, or knocks out, the ULK1 gene in the isolated cell.

36. The method of claim 29, wherein the effect of the expression product of the ULK1 gene is impaired by post-transcriptional gene interference.

37. The method of claim 36, wherein the post-transcriptional gene interference utilizes siRNA interference, microRNA interference, antisense RNA interference, or small molecule interference.

38. The method of claim 29, wherein the impairing of the effect of the expression product of the ULK1 gene results in an increase in production of a recombinant protein as compared to a cell in which the effect of the expression product of the ULK1 gene has not been impaired.

39. The method cell of claim 38, wherein the recombinant protein is a secreted protein, a membrane-anchored protein or an intracellular protein.

40. The method of any of claim 39, wherein the recombinant protein is an antibody.

41. The method of claim 40, wherein the increase in production of the recombinant protein is at least about 30%.

42. The method of claim 41, wherein the increase in production of the recombinant protein is about 50% to about 500%.
